(19) United States
(12) Patent Application Publication
(10) Pub. No.: US 2017/0071951 A1
(43) Pub. Date: Mar. 16, 2017

(54) Methionine Aminopeptidase Inhibitors for Treating Infectious Diseases

(71) Applicants: Omonike Arike Olaleye, Spring, TX (US); Jun O. Liu, Clarksville, MD (US); Sarah Finney John, Missouri City, TX (US); Adaugo Chimzurum Isichei, Sugar Land, TX (US)

(72) Inventors: Omonike Arike Olaleye, Spring, TX (US); Jun O. Liu, Clarksville, MD (US); Sarah Finney John, Missouri City, TX (US); Adaugo Chimzurum Isichei, Sugar Land, TX (US)

(73) Assignee: Texas Southern University, Houston, TX (US)

(21) Appl. No.: 15/366,078

(22) Filed: Dec. 1, 2016

Related U.S. Application Data
(63) Continuation of application No. 14/536,024, filed on Nov. 7, 2014.
(60) Provisional application No. 61/906,658, filed on Nov. 20, 2013.

(51) Int. Cl. A61K 31/5383; A61K 31/122; A61K 31/506; A61K 31/47; A61K 31/4409; A61K 31/519

(52) U.S. Cl. CPC A61K 31/5383; A61K 31/4409; A61K 31/519; A61K 31/506; A61K 31/47; A61K 31/122

(57) ABSTRACT

The present invention relates to methods for treating an infectious disease in a subject in need thereof via administration of a therapeutically effective amount of compounds described herein. The methods may utilize particular compounds, for example, a quinoline, a hydrazone, a quinone, or a pyrimidine derivative thereof or a pharmaceutical salt thereof.

### CROSS-REFERENCE TO RELATED APPLICATION

This continuation application claims benefit of priority under 35 U.S.C. §120 of pending non-provisional application U.S. Ser. No. 14/536,024, filed Nov. 7, 2014, which claims benefit of priority under 35 U.S.C. §119(e) of provisional application U.S. Ser. No. 61/906,658, filed Nov. 20, 2013, the entirety of both of which are hereby incorporated by reference.

### BACKGROUND OF THE INVENTION

Field of the Invention

The present invention generally relates to the fields of medicine and molecular biology of infectious diseases. In particular aspects, the field of the invention relates to particular compositions and methods for the treatment of diseases, such as Human Immunodeficiency Virus (HIV), Mycobacterium tuberculosis (Mtb), Gram-positive and Gram-negative bacterial infections, as well as parasitic infections.

Description of the Related Art

Infectious disease is the second leading cause of death worldwide, and the third leading cause of death in the United States of America. Particularly, Tuberculosis (TB) and Human Immunodeficiency Virus (HIV) remains the top two leading cause of mortality due to an infectious disease globally. The World Health Organization (WHO) estimates that of the 34 million cases of HIV, one third is also co-infected with latent tuberculosis. A lethal synergy exists between the two pathogens, Mycobacterium tuberculosis (Mtb) and HIV, which has led to the decline in the immune function of infected individuals and a rise in morbidity and mortality rates. Due to the emergence of drug resistant TB and HIV strains, drug-to-drug interactions, and increased drug toxicity, the therapeutic management of co-infected individuals remains a challenge.

A global rise in the incidence of multidrug-resistant (MDR), extensively drug-resistant (XDR), and totally drug-resistant (TDR) strains of M. tuberculosis and co-infected individuals has made it imperative to identify potent anti-mycobacterials with novel targets. While recent studies have provided important insights to the development of novel anti-bacterial and anti-viral drugs, discovery and development of a novel class of HIV-TB co-infection inhibitors that are efficacious and selective with improved pharmacologic profiles is vital. Therefore, anti-tuberculosis agents with a novel mechanism of action that also has an anti-HIV activity may help reduction in pill burden, reduction in the cost of treatment, and possibly increase patient compliance.

The prior art is deficient in the novel compositions and methods useful for the treatment of a variety of infectious diseases by developing selective anti-infective agents that shows selectivity for various Methionine aminopeptidases over human Methionine aminopeptidases. The present invention fulfills this longstanding need and desire in the art.

### SUMMARY OF THE INVENTION

The present invention is directed to a related method for treating an infectious disease in a subject in need thereof. The method comprises administering to the subject, in a pharmaceutically acceptable medium, a therapeutically effective amount of a methionine aminopeptidase inhibitor having the chemical structure Formula I:

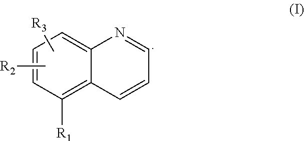

The R₁ substituent may be halogen. The R₂ and R₃ substituents independently may be halogen, OH, or —O(O)CCH₃, or R₂ and R₃ together form an N-substituted 1,3-oxazinanane. The compound may be a pharmaceutically acceptable salt or a stereoisomer thereof or a combination thereof.

The present invention also is directed to a related method for treating an infectious disease in a subject in need thereof. The method comprises administering to the subject, in a pharmaceutically acceptable medium, a therapeutically effective amount of methionine aminopeptidase (MetAP) inhibitor having the chemical structure Formula II:

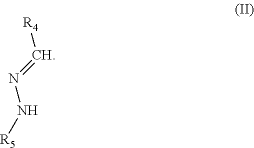

The R₄ substituent may be

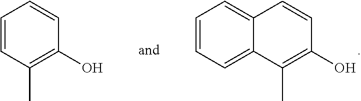

The R₅ substituent may be isonicotonyl group or

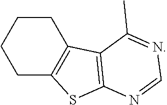

The compound may be a pharmaceutically acceptable salt or a stereoisomer thereof or a combination thereof.

The present invention is directed further to a related method for treating an infectious disease in a subject in need thereof. The method comprises administering to the subject, in a pharmaceutically acceptable medium, a therapeutically effective amount of methionine aminopeptidase (MetAP) inhibitor which is a quinone having the chemical structure Formula III:

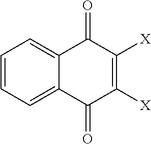

wherein X is a halogen such as chlorine or bromine.

The present invention is directed further to a methionine aminopeptidase (MetAP) inhibitors having the structures shown in compounds 1 to 10:

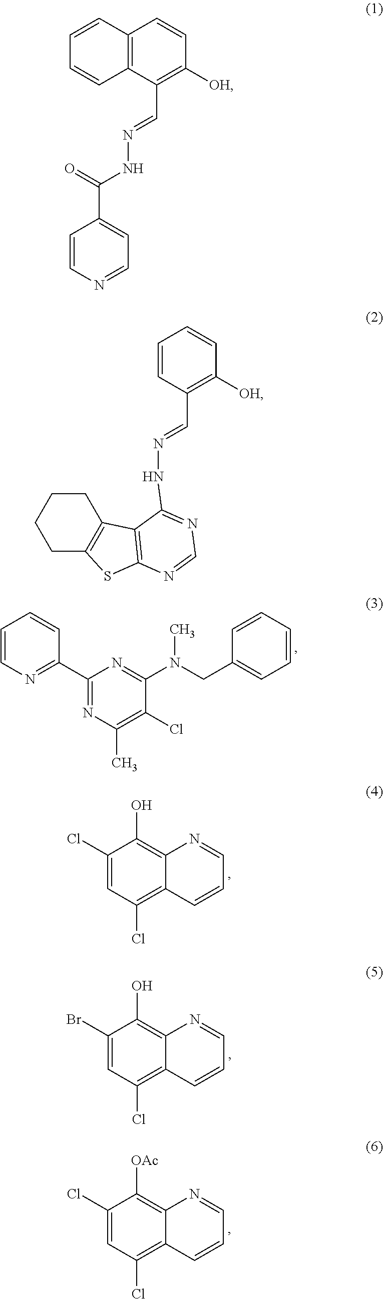

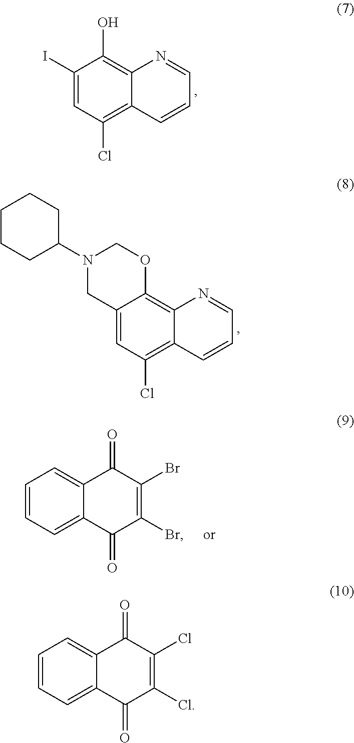

The present invention is directed further still to a methionine aminopeptidase (MetAP) inhibitor that is 5-chloro-7-iodoquinolin-8-ol; 7-bromo-5-chloroquinolin-8-ol; 5,7-dichloroquinolin-8-01; 5,7-dichloroquinolin-8-yl acetate; 6-chloro-3-cyclohexyl-3,4-dihydro-2H-[1,3]oxazino[5,6-h]quinolin; N-benzyl-5-chloro-N,6-dimethyl-2-(pyridin-2-yl)pyrimidin-4-amine; N′-((2-hydroxynaphthalen-1-yl) methylene)isonicotinohydrazide; 2-{(E)-[2-(5,6,7,8-tetrahydro[1]benzothieno[2,3-d]pyrimidin-4-yl) hydrazinylidene]methyl} phenol; 2,3-dichloronaphthalene-1,4-dione; or 2,3-dibromonaphthalene-1,4-dione.

The foregoing has outlined rather broadly the features and technical advantages of the present invention in order that the detailed description of the invention that follows may be better understood. Additional features and advantages of the invention will be described hereinafter, which form the subject of the claims of the invention. It should be appreciated by those skilled in the art that the conception and specific embodiment disclosed may be readily utilized as a basis for modifying or designing other structures for carrying out the same purposes of the present invention. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1C illustrate methionine aminopeptidases inhibitors as novel entities for targeting HIV and HIV/TB co-infection. Inhibition of HIV-1 replication by MetAP inhibitors (Compounds 1-3, 5 and 7) is depicted with virus controls (R1, R2, R3, R4, and R5) in FIG. 1A. Inhibition of HIV-1 replication by MetAP inhibitors (Compounds 1, 2, and 6) is depicted with virus positive and negative controls in FIG. 1B. Preliminary data for Inhibition of HIV-1 replication by Antimycobacterial compounds (Compound 1, INH (Isoniazid), PZA (Pyrazinamide)) is shown in FIG. 1C.

FIGS. 2A-2C illustrate the application of co-infection model to evaluate compound activities. ELISA analysis of HIV-1 p24 secretion into the media from infected PBMC (n=6 for DMSO, BCG, AZT groups and n=4 for groups with RIF) is shown in FIG. 2A. PBMCs were isolated from peripheral blood of healthy human donors, infected with mock (media), HIV-189.6 for 5 h and/or M. bovis BCG (tdtomato) for 1 h. Samples were treated with gentamicin (50 ng/ml) to eliminate extracellular bacteria, washed, and cultured for 7 days in media or with various drug treatments: AZT (5.0 μg/mL); RIF (5.0 μg/mL); INH (0.1 μg/mL); or DMSO (1%). A, ELISA analysis of HIV-1 p24 secretion into the media from infected PBMC, (n=6 for DMSO, BCG, AZT groups and n=4 for groups with RIF). FIG. 2B shows a CFU enumeration of BCG-infected PBMCs as affected by single and dual drug treatments (n=5 except RIF and INH groups where n=2-3 were used). FIG. 2C shows intracellular growth of M. bovis BCG normalized to growth in the absence of HIV-1 infection or drug treatment per individual donor. The dotted grey line indicates baseline growth of BCG in the absence of HIV-1 or drug treatment. Values shown are the means±SEM. Statistically significant decreases in p24 secretion (FIG. 2A) or CFU (FIGS. 2B-2C) due to drug treatment compared to treatment with vehicle (DMSO) are designated as follows:*, p<0.05; **, p<0.01, ***, p<0.001. A statistically significant increase in CFU due to HIV-1 co-infection is designated by ††, p<0.01.

FIGS. 3A-3E illustrate cell viability following infection and drug exposure in co-infected PBMCs. Flow cytometric analysis of side scatter and forward scatter characteristics of isolated cells and representative flow cytometry plots of the cell death determined by flow cytometric analysis of live/dead fixable aqua marker of cell death is shown in FIGS. 3A-3D. FIG. 3E shows summarized data of cell viability as affected by carrier (DMSO), infection with HIV, BCG, or both in carrier, and exposure to standard drug compounds (Azidothymidine, AZT, 5 μg/ml; Isoniazid, INH, 0.1 μg/ml, and Rifampicin, RIF, 5 μg/ml). Values shown are the means±SEM of results from 3 individual donors, performed in duplicate.

FIGS. 4A-4H illustrate flow cytometric analysis of drug treatment effects on HIV/BCG co-infection assay. Flow cytometric analysis of side scatter and forward scatter characteristics of isolated macrophages (Gate 1) and a representative plot showing the % of BCG (PE-Texas Red) and HIV (FITC) co-infected macrophages in the selected (Gate 1) population is shown in FIGS. 4A-4D. FIGS. 4E-4H shows representative plots demonstrating percentage of macrophages positive for the intracellular BCG (tdtomato fluorescence) or intracellular HIV (FITC fluorescence) as affected by treatment from 3 individual donors.

FIG. 5 illustrates an experimental design of BCG/HIV PBMC co-infection assay. The experimental flow chart for isolation, infection, and assessment of co-infected PBMC following treatment with anti retroviral and antimycobacterial drug regimens is shown.

FIGS. 6A-6B illustrates anti-leishmanicidal activity of MetAP inhibitors in L. major promastigotes wild type (FIG. 6A) and L. major overexpressing MetAP1 (FIG. 6B).

FIGS. 7A-7B Depicts E. faecalis MetAP1 as an antibacterial target. High-throughput screening of compounds against E. faecalis MetAP1 is shown in FIG. 7A. Purification of MetAP from E. faecalis is shown in FIG. 7B.

FIGS. 8A-8E Depicts biochemical characterization of MetAP from E. faecalis. FIG. 8A depicts the velocity versus substrate concentration plot for MetAP from E. faecalis to determine kinetic constants. FIG. 8B depicts relative MetAP activity versus temperature plot to determine an optimal temperature for EfMetAP1. FIG. 8C depicts relative MetAP activity versus pH. FIGS. 8D-8E depict metal dependence of EfMetAP1. FIG. 8D depicts relative MetAP activity versus Cobalt (II) chloride concentration. FIG. 8E depicts relative MetAP activity versus Magnesium (II) chloride concentration.

FIG. 9 shows C. elegans survival curve and determination of effective concentration in non-infected C. elegans where nematodes were treated with compound 5 at concentrations of 4.0 μg/mL to 4×10⁻⁴ μg/mL.

FIGS. 10A-10D show C. elegans survival curve and determination of Minimum Protective Concentration (MPC). C. elegans infected with OG1RF strain of E. faecalis were treated with compound 5 at concentrations of 4×10⁻⁴ μg/mL to 4.0×10⁻³ μg/mL (FIG. 10A), 4×10⁻⁷ μg/mL to 4.0×10⁻⁵ μg/mL (FIG. 10B), 4×10⁻¹⁰ μg/mL to 4.0×10⁻⁵ μg/mL (FIG. 10C), and 4×10⁻¹² μg/mL to 4.0×10⁻¹¹ μg/mL (FIG. 10D).

FIG. 11 shows C. elegans survival curve when infected with V583 and determination of Minimum Protective Concentration (MPC) in VRE. As shown, C. elegans are treated with compound 5, at concentrations of 4×10⁻¹² μg/mL to 4.0×10⁻⁹ μg/mL.

FIG. 12 shows the bacterial load in E. faecalis infected nematodes after tetracycline treatment at 10 μg/mL and compound 5 treatment at concentrations of 4×10⁻⁵ μg/mL and 4×10¹¹ μg/mL. Error bars represent standard deviations calculated from three independent experiments. ** (P<0.01), *** (P<0.0001). Statistical differences were determined by unpaired t-test using GraphPad Prism version 5.0. P values<0.05 were considered to be statistically significant.

FIG. 13 is a preliminary screen of compounds 4 and 5 against Pseudomonas aeruginosa (PA14) in LB media at concentrations ranging from 2.5 to 40 μg/mL.

FIG. 14. is a preliminary screen of compounds 4 and 5 against Pseudomonas aeruginosa (PA14) in SK media at concentrations ranging from 2.5 to 40 μg/mL.

### DETAILED DESCRIPTION OF THE INVENTION

### General Embodiments of the Invention

Methionine aminopeptidase (MetAP), a metalloprotease that catalyzes the removal of the initiating N-terminal methionine from proteins and polypeptides, is a promising and attractive drug target. N-terminal methionine excision is required for post-translational modifications, stability, localization and maturation of a large number of proteins and is therefore an essential process. In specific embodiments in this invention, small-molecule compounds are evaluated as potent inhibitors of MetAP for targeting bacterial, viral and parasitic infections. In one embodiment of the present invention, there is provided a method for treating an infectious disease in an subject in need thereof, comprising administering to the subject, in a pharmaceutically acceptable medium, a therapeutically effective amount of methionine aminopeptidase inhibitor.

In a preferred aspect of this method of present invention, the methionine aminopeptidase inhibitor is a quinoline having the chemical structure shown in Formula I:

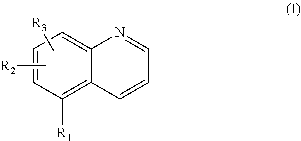

wherein R₁ is a halogen; and R₂ and R₃ independently are halogen, OH or —OC(O)CH₃, or R₂ and R₃ together form an N-substituted 1,3-oxazinanane; or a pharmaceutically acceptable salt or a regioisomer thereof or a combination thereof.

In another preferred aspect of this method of present invention, the methionine aminopeptidase inhibitor is a hydrazone having the chemical structure shown in Formula II:

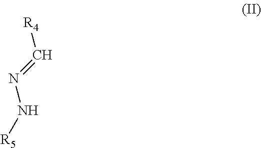

wherein R₄ is

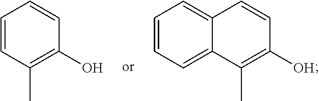

and R₅ is isonicotonyl group or

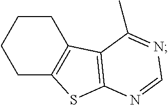

or a pharmaceutically acceptable salt thereof.

In another preferred aspect of this method of present invention, the methionine aminopeptidase inhibitor is a quinone having the structure shown in Formula III:

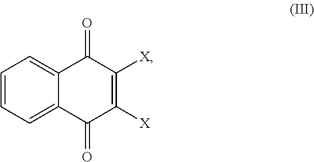

wherein X is a halogen such as chlorine or bromine.

In all embodiments and aspects thereof of the present invention, the infectious disease may be Human Immunodeficiency Virus, Mycobacterium tuberculosis, a Gram-positive bacterial infection, a Gram-negative bacterial infection, a parasitic infection or a combination thereof. For example, the subject is co-infected with Human Immunodeficiency Virus and Mycobacterium tuberculosis. The Gram-positive and/or Gram-negative bacterial infection comprises a nosocomial infection. The parasitic infection is Leishmaniasis.

Preferred compounds of present invention are 5-chloro-7-iodoquinolin-8-ol; 7-bromo-5-chloroquinolin-8-ol; 5,7-dichloroquinolin-8-ol; 5,7-dichloroquinolin-8-yl acetate; 6-chloro-3-cyclohexyl-3,4-dihydro-2H-[1,3]oxazino[5,6-h]quinolin; N-benzyl-5-chloro-N,6-dimethyl-2-(pyridin-2-yl)pyrimidin-4-amine; N′-((2-hydroxynaphthalen-1-yl)methylene)isonicotinohydrazide; 2-{(E)-[2-(5,6,7,8-tetrahydro[1]benzothieno[2,3-d]pyrimidin-4-yl)hydrazinylidene]methyl}phenol; 2,3-dichloronaphthalene-1,4-dione; or 2,3-dibromonaphthalene-1,4-dione.

In another embodiment of the present invention, there is provided a method for treating an infectious disease in a subject in need thereof, the method comprising administering to the subject, in a pharmaceutically acceptable medium, a therapeutically effective amount of a methionine aminopeptidase inhibitor having the chemical structure shown in Formula II:

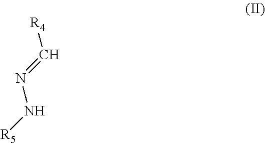

wherein R₄ is

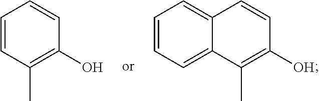

and R₅ is isonicotonyl group or

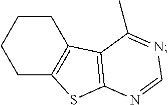

or a pharmaceutically acceptable salt or a regioisomer thereof or a combination thereof. In a preferred aspect of this embodiment, the infectious disease may be Human Immunodeficiency Virus, Mycobacterium tuberculosis or a combination thereof or a parasitic infection. Examples of Mycobacterium tuberculosis are Wild-type M. tuberculosis, Dormant M. tuberculosis or multi-drug resistant M. tuberculosis.

In this embodiment and aspects thereof, a therapeutically effective amount of the compound selectively inhibits the bacterial methionine aminopeptidase over a human methionine aminopeptidase. The selectivity for Mycobacterium tuberculosis methionine aminopeptidase over human methionine aminopeptidase is about 20 fold to about 50 fold or more depending on the inhibitor. The Mycobacterium tuberculosis methionine aminopeptidase is MtMetAP1a or MtMetAP1c. The human methionine aminopeptidase is HsMetAP1 or HsMetAP2. In another aspect of this embodiment, the parasitic infection is Leishmaniasis and the methionine aminopeptidase is L. major MetAP1 (LmMetAP1).

The chemical structure of methionine aminopeptidase inhibitor is shown in compounds 1 to 3:

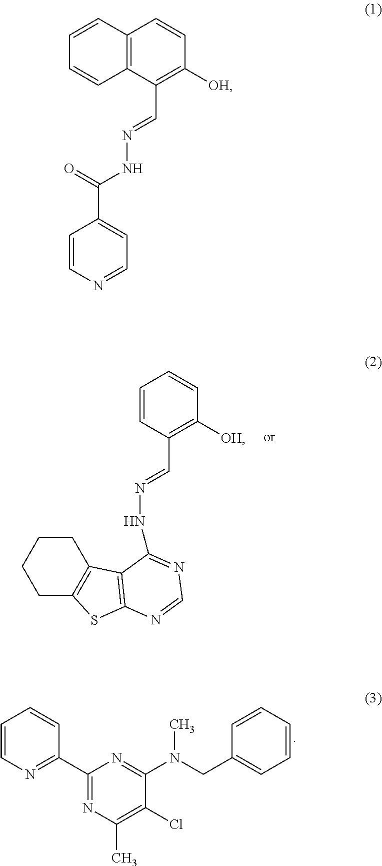 [SMILES: O=C(N/N=C/c1c(O)ccc2ccccc12)c1ccncc1 ; Oc1ccccc1/C=N/Nc1ncnc2sc3c(c12)CCCC3 ; "or" ; Cc1nc(-c2ccccn2)nc(N(C)Cc2ccccc2)c1Cl]

In another embodiment of the present invention, there is provided a method for treating an infectious disease in a subject in need thereof, the method comprising administering to the subject, in a pharmaceutically acceptable medium, a therapeutically effective amount of a methionine aminopeptidase inhibitor having the chemical structure shown in Formula (I):

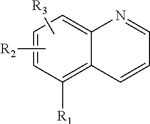

wherein R₁ is halogen; and R₂ and R₃ independently are halogen, OH, or —O(O)CCH₃, or R₂ and R₃ together form an N-substituted 1,3-oxazinanane; or a pharmaceutically acceptable salt or a regioisomer thereof or a combination thereof.

Preferred quinolone compounds of this embodiment are compounds 4 to 8:

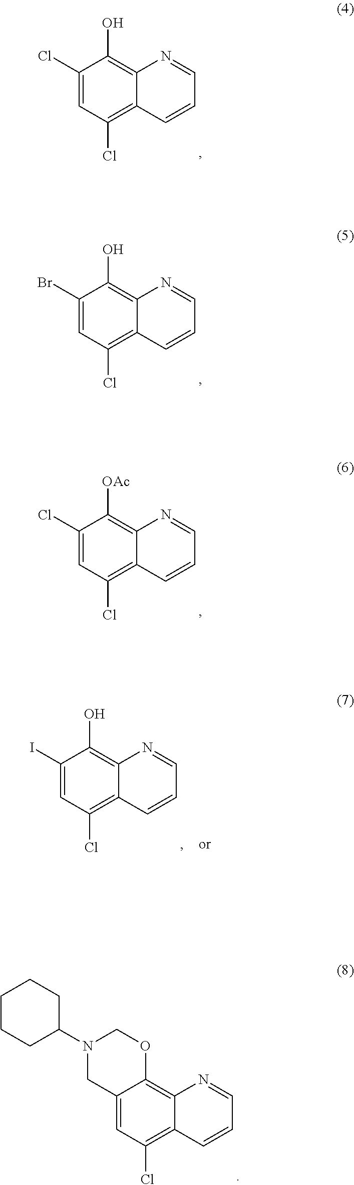 [SMILES: Oc1c(Cl)cc(Cl)c2cccnc12 ; Oc1c(Br)cc(Cl)c2cccnc12 ; CC(=O)Oc1c(Cl)cc(Cl)c2cccnc12 ; Oc1c(I)cc(Cl)c2cccnc12 ; Clc1cc2c(c3ncccc13)OCN(C1CCCCC1)C2]

In a preferred aspect of this embodiment, the subject is infected with a Gram-positive or/and Gram negative bacterium. In another preferred aspect of this embodiment, the cells, tissue culture or subject is infected with Human Immunodeficiency Virus or Mycobacterium tuberculosis or a combination thereof.

The Gram-negative bacterium is Pseudomonas areuginosa, the Gram-positive bacterium is Enterococcus faecalis (E. faecalis) bacterium and the methionine aminopeptidase is E. faecalis MetAP1.

In another aspect of this embodiment, the Gram-positive and Gram-negative bacterial infection independently comprise a nosocomial infection. The nosocomial infection, for example, is bacteremia, endocarditis, pneumoniasurgical site infections or a urinary tract infections. In a specific aspect, the bacterium is a vancomycin-resistant Enterococcus (VRE) bacterium.

In an embodiment of present invention, a methionine aminopeptidase inhibitor having the chemical structure shown in compounds 1-10:

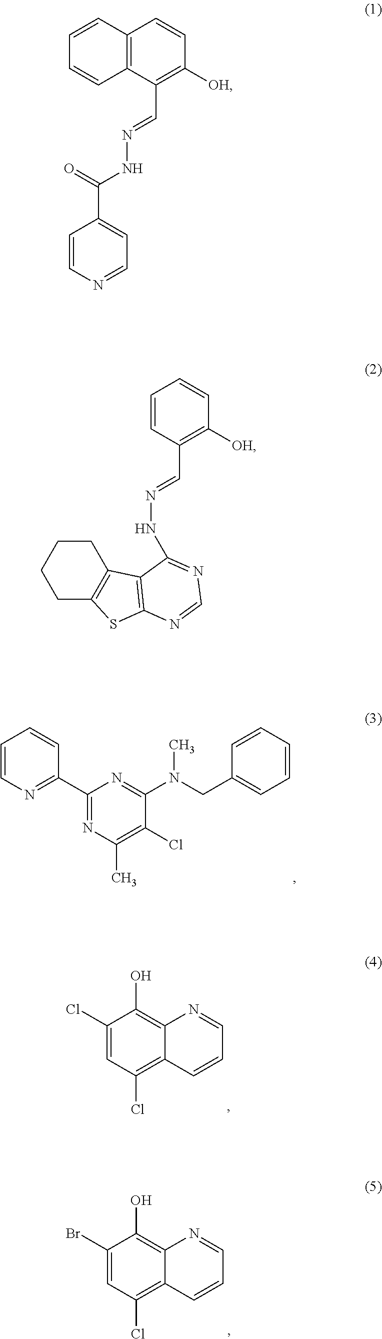 [SMILES: O=C(N/N=C/c1c(O)ccc2ccccc12)c1ccncc1 ; Oc1ccccc1/C=N/Nc1ncnc2sc3c(c12)CCCC3 ; Cc1nc(-c2ccccn2)nc(N(C)Cc2ccccc2)c1Cl ; Oc1c(Cl)cc(Cl)c2cccnc12 ; Oc1c(Br)cc(Cl)c2cccnc12]

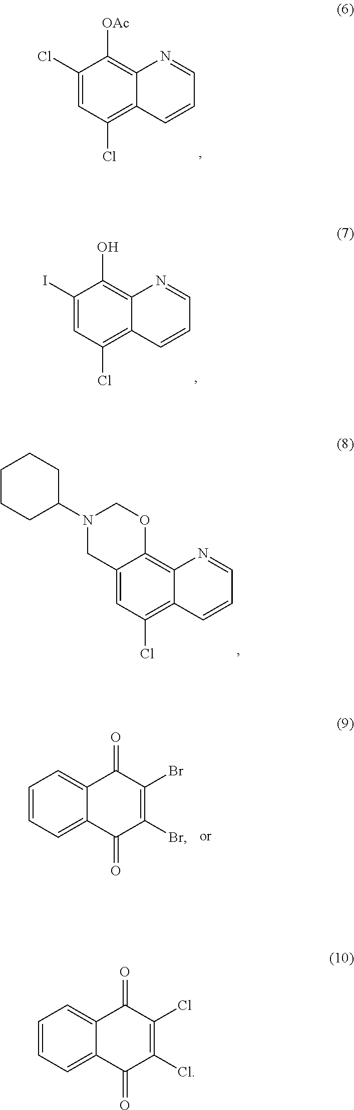 [SMILES: CC(=O)Oc1c(Cl)cc(Cl)c2cccnc12 ; Oc1c(I)cc(Cl)c2cccnc12 ; Clc1cc2c(c3ncccc13)OCN(C1CCCCC1)C2 ; O=C1C(Br)=C(Br)C(=O)c2ccccc21 ; "or" ; O=C1C(Cl)=C(Cl)C(=O)c2ccccc21]

In all these embodiments and aspects thereof a person having ordinary skill in this art could readily determine a useful dose of a compound of the present invention depending upon the indication to be treated or the outcome desired. The compounds of the present invention showed minimum inhibitory concentrations (MICs) in the low micro molar range. Typically, the compound is administered in a dose ranging from about 0.05 μg/mL to about 50 μg/mL, preferably from about 0.2 μg/mL to about 10 μg/mL.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

As used herein, the term “subject” shall refer to a human or other animal, for example, a mammal.

The term “halogen” includes iodine, bromine, chlorine and fluorine.

The term “substituted” shall be deemed to include multiple degrees of substitution by a substituent. A substitution occurs where a valence on a chemical group or moiety is satisfied by an atom or functional group other than hydrogen. In cases of multiple substitutions, the substituted compound can be independently substituted by one or more of the disclosed or claimed substituent moieties, singly or plurally. By independently substituted, it is meant that the (two or more) substituents can be the same or different.

The term “isomers” as used herein is a form of isomerism in which molecules with the same molecular formula have bonded together in different orders and have different structural formula. Position isomers are structural isomers in which a functional group or a substituent changes position on a parent structure.

The term “effective concentration (EC₅₀)” as used herein is defined as the concentration at which 50% of the infected treated groups are statistically significantly different from the infected (DMSO) mock-treatment group (p<0.05).

The term “lethal concentration (LC₅₀)” as used herein is defined as the concentration at which 50% of non-infected treated groups are statistically significantly different from the non-infected (DMSO) mock-treatment group (p<0.05).

The term “minimum protective concentration (MPC)” as used herein is defined as the minimum concentration required to obtain at least 25% survival of the nematodes.

The term “pharmaceutically acceptable salt” refers herein to a salt of a compound that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The present invention also includes protected derivatives of compounds disclosed herein. For example, when compounds of the present invention contain groups such as hydroxyl, amine or carbonyl, these groups can be protected with a suitable protecting group. A list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of the present invention can be prepared by methods well known in the art.

### Pharmaceutical Preparations

Pharmaceutical compositions of the present invention comprise an effective amount of one or more compositions of the invention (and additional agent, where appropriate) dissolved or dispersed in a pharmaceutically acceptable carrier. The phrases “pharmaceutical or pharmacologically acceptable” refers to molecular entities and compositions that do not produce an adverse, allergic or other untoward reaction when administered to a human or animal, as appropriate. The preparation of a pharmaceutical composition that contains at least one MetAP inhibitor or additional active ingredient will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the pharmaceutical compositions is contemplated.

The MetAP inhibitors may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens, e.g., methylparabens, and propylparabens, chlorobutanol, phenol, sorbic acid, thimerosal or combinations thereof. In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include MetAP inhibitors, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

One of ordinary skill in the art would be familiar with the range of techniques that can be employed for dispersing a composition in a lipid vehicle. For example, the MetAP inhibitor or derivative thereof may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

### Kits of the Invention

Any of the compositions described herein may be comprised in a kit. In a non-limiting example, the kit comprises a composition suitable for treatment and/or prevention of one or more infectious diseases. In other embodiments of the invention, the kit comprises one or more apparatuses to obtain a sample from an individual. Such an apparatus may be one or more of a swab, such as a cotton swab, toothpick, scalpel, spatula, syringe, and so forth, for example. In another embodiment, an additional compound is provided in the kit, such as an additional compound for treatment and/or prevention of a infectious disease. Any compositions that are provided in the kits may be packaged either in aqueous media or in lyophilized form, for example. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

### Primary High-Throughput Screening

A primary high throughput screen was conducted using MAP-C2 microplate processor (Titertek Instruments, Inc., Huntsville, Ala. USA). A library of 175,000 compounds at 30 μM were assayed in a 384 well plates using a dipeptide chromogenic substrate, methionine-proline coupled to p-Nitroaniline (Met-Pro-pNA). Each compound was dissolved in DMSO and stored at −20° C. until use. The total reaction volume was 50 μL, and each reaction contained 40 mM HEPES buffer (pH 7.5), 100 mM NaCl, 100 mg/mL BSA, 0.1 U/mL ProAP, 1.5 mM CoCl₂, 600 mM substrate (Met-Pro-pNA), and 252 nM MtMetAP1c. The enzyme was pre-incubated with compounds for 20 minutes at room temperature followed by addition of 600 mM substrate. The reaction was then incubated at room temperature for 30 min and monitored at 405 nm on a spectrophotometer. Compounds that showed greater than 30-40% inhibition were chosen as “hits”.

### Determination of IC₅₀ of MetAP Inhibitors

The concentration needed for 50% enzyme inhibition (IC₅₀) was determined in 96-well plates at final concentrations ranging from 100 μM to 300 nM for MetAP inhibitors. The enzyme was pre-incubated with compounds for 20 minutes at room temperature followed by addition of substrate. The reaction was incubated at room temperature for 30 min and monitored at 405 nm on a spectrophotometer. The background hydrolysis was subtracted and the data was fitted to a four-parameter logistic (variable slope) equation using GraphPad Prism software.

### HIV and Mycobacterial Isolates

The HIV isolate used in this study was a dual tropic (R5 and X4) strain (HIV-1 89.6), received gratis from Dr. Monique Ferguson (UTMB Galveston). A Mycobacterium bovis (M. bovis) BCG strain expressing a red fluorescent protein (tdtomato, with a spectrum similar to Texas Red) was received from Dr. Jeffrey Cirillo (Texas A&M Health Sciences Center). M. bovis BCG working stock used for the studies were cultured using standard BCG growth media which was made from DIFCO 7H9 Broth (Becton Dickinson, San Diego, Calif.). Briefly, cultures were grown in a shaker at 100 rpm, 37° C. for 10-14 days and kanamycin (50 ng/ml) was added to the stock once in 5 days to maintain the plasmid expressing tdtomato. When the OD value of the growing stock reached 0.8-1.0, the stocks were frozen in the storage medium (long term storage) or PBS (for infections) at −80° C.

### Cell Preparation and Activation

Peripheral blood was obtained from healthy donors as approved by the UTMB Institutional Review Board. Peripheral blood mononuclear cells (PBMC) were isolated from heparinized peripheral blood using Accuprep (Accurate chemicals, New York, N.Y.) and density centrifugation. An RBC Lysis buffer (Sigma, St. Louis, Mo.) was used to remove any remaining red blood cells according to the manufacturer recommendation. Cell viability of the isolated PBMC population was determined by using trypan blue exclusion and by flow cytometry using the live/dead aqua cell viability assay (Invitrogen, New York, N.Y.). Cells were cultured in cRPMI at 106 cells/ml of media in 5% CO₂ at 37 C (Hogg et al. J Leukoc Biol, 86:1191, 2009). In some experiments, peripheral blood monocytes were isolated by magnetic bead-conjugated antibody separation using AutoMacs (Gonzales et al. Infect Immun, 80:234, 2012) and used to derive macrophages following 5-6 days of culture with 50 ng/ml of recombinant human M-CSF.

### Flow Cytometry

The mAbs against FITC-conjugated HIV p24 was purchased from Beckman Coulter (Indianapolis, Ind.). Following infection and treatment, PBMC or monocyte-derived macrophages were harvested at appropriate time points for flow cytometric analysis. After harvest, cells were incubated with CD16/CD32 Fc Block (BD Biosciences, San Jose, Calif.,) to reduce non-specific binding of antibodies. Cells were permeabilized using the BD Cytofix/Cytoperm kit (BD Pharmingen, CA), then labeled with a FITC-conjugated antibody specific to HIV-1 (KC-67, Beckman-Coulter) as previously described (Hogg, et al. 2009). Samples were finally incubated for 48 hours in 4% formaldehyde (Polysciences Inc, Warrington, Pa.) and diluted in PBS prior to acquisition. A total of 50,000 gated events, based on expected leukocyte side scatter/forward scatter characteristics, were collected using a BD LSR II (Fortessa) flow cytometer (BD Biosciences). Analysis of data was performed by FCS Express 4 (De Novo, Los Angeles, Calif.) software. To control for background and to establish thresholds for gating positive cells, an isotype-matched FITC-labeled antibody was used.

### ELISA

The levels of secreted HIV p24 in culture supernatants of purified CD14+ or PBMC was measured at day 7 using an ELISA kit purchased from Zeptometrix (Buffalo, N.Y.). Secreted protein was converted to pg/ml based on the standard curve generated from known p24 standards included in the kit as recommended by the manufacturer.

### CFU Enumeration

Cells were disrupted with 0.067% SDS and lysates used to determine bacterial growth following culture with control or standard compounds. The bacterial load was measured by colony forming unit (CFU) enumeration by limiting dilutions and determining growth on selective agar (7H11).

### Determination of Inhibitory Concentrations in HIV-1 Infected Peripheral Blood Mononuclear Cells (PBMC)

Peripheral blood mononuclear cells (PBMC) were isolated from healthy human volunteers and cultured in cRPMI. The PBMC cells were stimulated with PHA-P for 1-3 days. The cells were plated in a 24 well plate and infected with HIV-1 (89.6 strain) for 24 hours at 30° C. and 5% CO₂. The cells were washed with antibiotic free cRPMI media to remove extracellular HIV p24 antigen. MetAP inhibitor (Compounds 1 to 8) was added to the 24 well plate at final concentrations ranging from 30 μM to 300 nM to give a total assay volume of 1 mL. The cells were incubated at 30° C. and 5% CO₂ for 7 days. The cells were harvested and the supernatant was analyzed for the amount of p24 antigen using the RetroTek HIV-1 p24 ELISA kit (Zeptometrix, Buffalo, N.Y.). Azidothymidine (AZT, 10 μM), DMSO, and a blank (drug free) were used as controls in this experiment.

Determination of Minimum Inhibitory Concentration in Replicating M. tuberculosis

Compounds were serial diluted in DMSO and added to 7H9 broth, 2% Glycerol, 0.05% Tween 80, and 10% albumin/dextrose complex (ADC) at final concentrations ranging from 50 μg/mL-0.05 μg/mL. A culture of M. tuberculosis CDC 1551 was grown till O.D.=1.0 and a 1/100 dilution is done. The tubes containing the test agent with 0.1 mL of bacterial culture was inoculated yielding a total assay volume of 5 mL. DMSO (negative control), Isoniazid (positive control), and drug free media (blank) were used as controls to determine minimum inhibitory concentration of the compounds.

Activity Against Dormant M. tuberculosis

The activity of the compounds in aged non-growing M. tuberculosis was achieved using a perister model at concentrations 0.5 to 100 mM and monitored for three weeks. Briefly, 2 month old M. tuberculosis H37Ra culture was grown in 7H9 medium (Difco) with 10% albumin dextrose catalase (ADC) and 0.05% Tween 80. The bacterial culture is then resuspended in 7H9 medium (pH 5.5) without ADC and an innocula was used for determining the activity of the compounds (e.g. 2-hydroxy-1-naphthylaldehyde isonicotinoyl hydrazone) for persister bacilli. The compound was diluted from a 10 mM (in DMSO) to 10 μM (final concentration) and incubated with the bacilli in 200 mL 7H9 medium (pH 5.5) without ADC in a 96-well plate for three days without shanking under 1% oxygen in a hypoxic chamber. This assay was performed in duplicates with Rifampin (5 mg/mL). Following a 3 day drug exposure, bacilli viability was determined by adding 20 mL of 1 mg/mL of XTT (2,3-bis-(2-methoxy-4-nitro-5-sulfophenyl)-2H-tetrazolium-5-carboxanilide) and incubated at 37° C. for up to 7 days. The 96 well plates were then read at OD 485 nm.

### Example 2

### Effects of Compounds 1-8 on HIV-1 p24 Antigen Levels

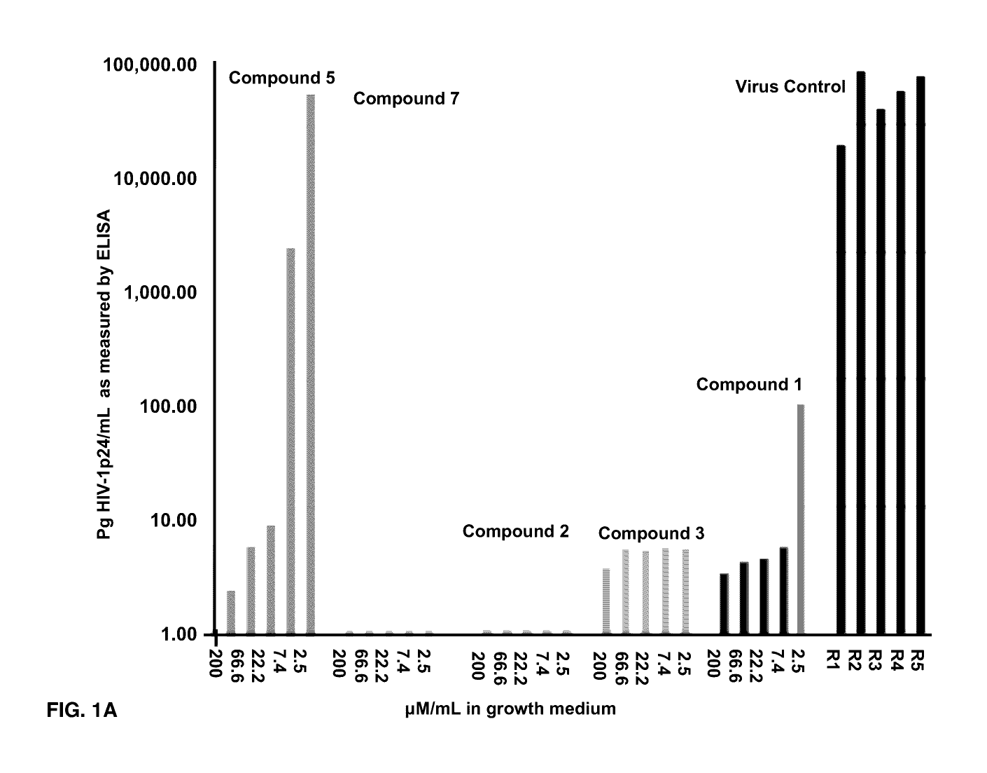

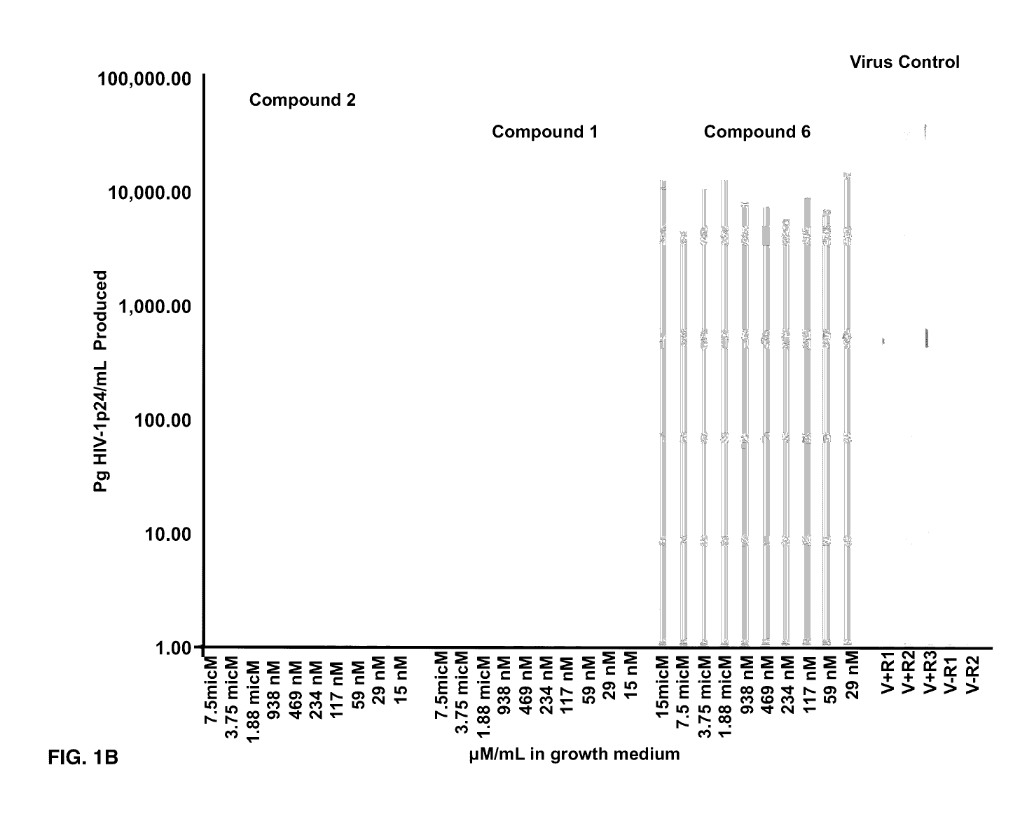

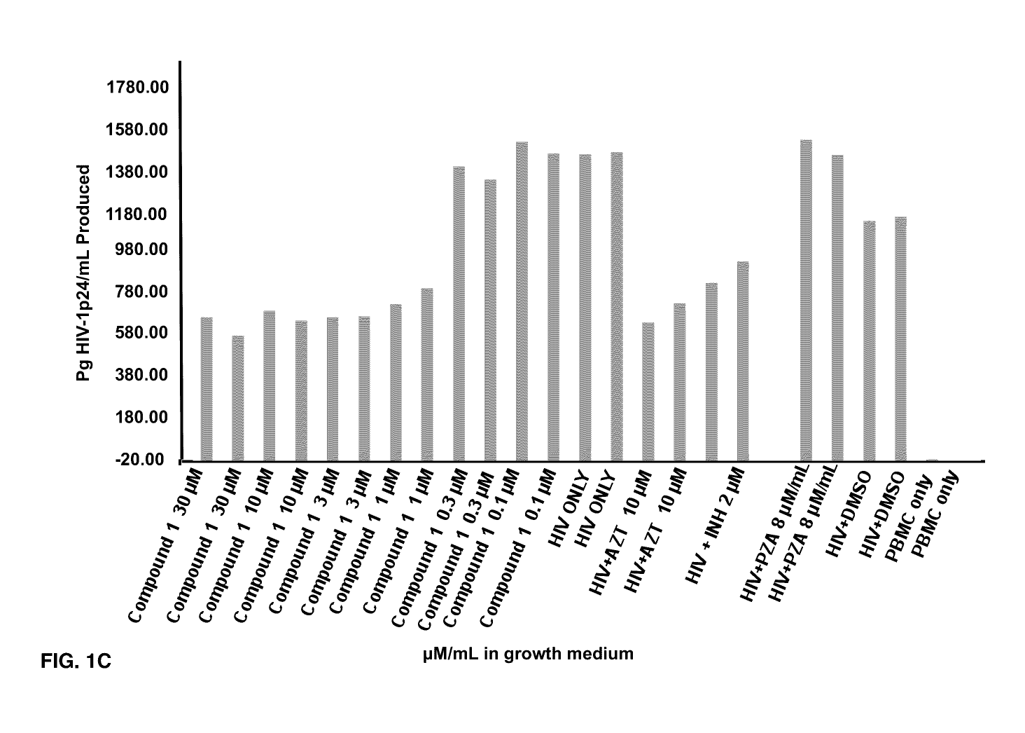

2-hydroxy-1-naphthylaldehyde isonicotinoyl hydrazone (1) is known to inhibit HIV-1 transcription with IC₅₀=2 μM by inhibiting proteins necessary for the cell cycle progression (Debebe et. al. 2007). Serendipitously 2-hydroxy-1-naphthylaldehyde isonicotinoyl hydrazone was identified in the M. tuberculosis screen and found that it also inhibited the production of HIV-1 p24 antigen in a dose response manner (FIGS. 1A-1B) and had activity similar to the positive control AZT. Moreover, compound 1 is structurally similar to a known anti-TB drug—Isoniazid. In the primary screen, it was found for the first time that Isoniazid has anti-HIV 1 activity. The results show that the concentration of Isoniazid required for 50% inhibition of HIV1 p24 produced is greater than 2 μM (FIG. 1C). Interestingly, this preliminary result is comparable to the anti-HIV IC50 for compound 1, its structural analogue (FIG. 1C). Furthermore, another anti-TB drug did not show any HIV1 activity (FIG. 1C). These results suggest that compound 1 and Isoniazid are potential compounds for the development of novel chemotherapeutic entities for the therapeutic management of TB-HIV co-infected individuals.

Inhibitory effects of compounds 2, 3, 5, 6, and 7 on HIV-1 infectivity was investigated by quantifying the level of p24 antigen produced using the ELISA (FIGS. 1A-1D). Compounds 2 to 3 inhibited p24 antigen productions in a dose dependent manner (FIGS. 1A-1B). For both compounds 4 and 8, the primary screen revealed that the IC50 is greater than 3.75 μM (data not shown). The inhibitory concentration equivalent to negative control (blank) was found to be 234.0 nM and 274.0 nM for compounds 2 and 1 respectively (FIG. 1B).

### Example 3

### Selectivity of 2-Hydroxy-1-Naphthylaldehyde Isonicotinoyl (1) Hydrazone for MtMetAP Enzymes Over HsMetAPs

2-hydroxy-1-naphthylaldehyde isonicotinoyl hydrazone (1) inhibited MtMetAP1a and MtMetAP1c having IC₅₀ values in the lower micro molar concentrations (Table 2). Further, compound 1 showed greater selectivity for MtMetAPs in comparison to HsMetAP1 and HsMetAP2. Compound 1 has at least 50 fold selectivity for MtMetAPs than HsMetAP1 and at least 20 fold selectivity for MtMetAPs than HsMetAP2. The selectivity for the MtMetAPs over HsMetAPs emphasizes the potential of compound 1 to selectively target the pathogen without leading to toxic effects for the host. It has been established in recent years that HIV-1 uses host molecular machinery for the N-terminal modification of several viral proteins. One such modification is the N-myristoylation of Nef, a HIV-1 accessory protein. Myristic acid is added to the protein by human N-myristoyltransferase-1 (NMT1). However, for NMT1 to carry out this process, MetAP must catalyze the removal the initiation methionine. Without the myristoylation of Nef by NMT, optimized viral replication and AIDS progression can be hindered. Therefore, finding one drug that targets MetAP can be effective in the suppression of HIV and TB leading to developing novel anti-infectives for the therapeutic management of co-infected individuals.

Minimum Inhibitory Concentration of MetAP Inhibitors on Replicating M. tuberculosis

The effect of the compound 1 in replicating M. tuberculosis culture was determined. The Minimum Inhibitory Concentration (MIC) was found to be less than 10.0 μg/mL (Table 1). The potency of compound 1 was agreeable to the relative activity against MtMetAP1a and MtMetAP1c in culture. Similarly, the potent effects of compound 2 in replicating M. tuberculosis culture and the Minimum Inhibitory Concentration (MIC) was determined to be less than 10.0 μg/mL. (Table 2). The potent effects of compound 3 in replicating M. tuberculosis culture and the Minimum Inhibitory Concentration (MIC) was determined to be less than 5.0 μg/ml.

TABLE 1
Determination of Minimum Inhibitory Concentration of MetAP in M. tuberculosis

| | | Wild-type M. |
|---|---|---|
| | | tuberculosis |
| | | Strain CDC1551 |
| Compound | MetAP IC₅₀ (μM) | (μg/mL) |
| 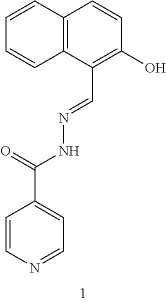 [SMILES: O=C(N/N=C/c1c(O)ccc2ccccc12)c1ccncc1] | MtMetAP1a: 4.9 MtMetAP1c: 5.3 HsMetAP1: 315.5 HsMetAP2: 145.6 | <10.0 |
| 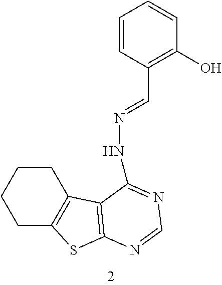 [SMILES: Oc1ccccc1/C=N/Nc1ncnc2sc3c(c12)CCCC3] | MtMetAP1a: <30.0 MtMetAP1c: <30.0 | <10.0 |
| 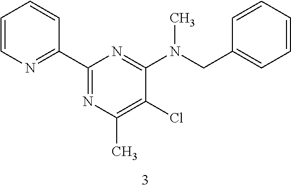 [SMILES: Cc1nc(-c2ccccn2)nc(N(C)Cc2ccccc2)c1Cl] | not determined | <5.0 |

Effects of 2-Hydroxy-1-Naphthylaldehyde Isonicotinoyl Hydrazone Against Dormant M. tuberculosis and Multi-Drug Resistant Tuberculosis

Minimum Inhibitory Concentration (MIC) of compound 1 and compound 2 on aged non-growing M. tuberculosis was determined (Table 2). At concentration of 1.82 μg/mL, compound 1 perturbed the growth of aged non-growing Mtb bacilli and inhibited the color development of redox dye XTT. Inhibitory effects of compounds 1-6, 9 and 10 on drug resistant strain M. tuberculosis HN 3409 in vivo were conducted for 29 days and the first preliminary results are outlined in Table 3 (these set of experiments are currently being repeated). The standards used for comparison where Linezolid and Kanamycin both at 5 μg/mL.

TABLE 2
Effects of Compounds 1 and 2 against dormant M. tuberculosis Strain
H37Ra

| Compound | H37Ra (μM) |
|---|---|
| 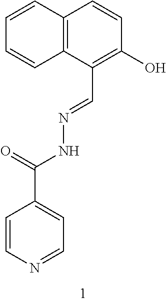 [SMILES: O=C(N/N=C/c1c(O)ccc2ccccc12)c1ccncc1] | 6.25 |
| 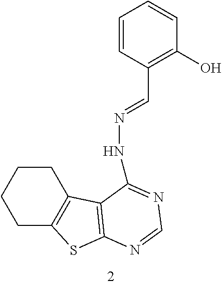 [SMILES: Oc1ccccc1/C=N/Nc1ncnc2sc3c(c12)CCCC3] | 25.0 |

TABLE 3
Preliminary results of effects of Compounds 1-6, 9 and 10 against
dormant Multi-Drug Resistant Tuberculosis Strain HN3409

| | HN3409 |
|---|---|
| Compound | (μg/mL) |
| 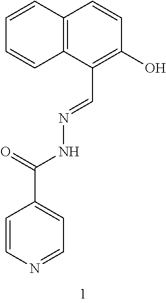 [SMILES: O=C(N/N=C/c1c(O)ccc2ccccc12)c1ccncc1] | >25.0 |
| 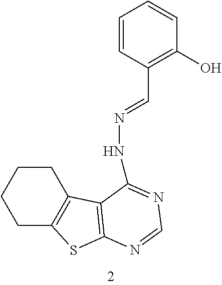 [SMILES: Oc1ccccc1/C=N/Nc1ncnc2sc3c(c12)CCCC3] | >25.0 |
| 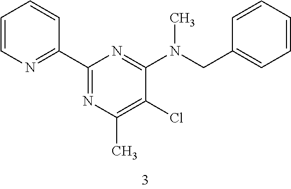 [SMILES: Cc1nc(-c2ccccn2)nc(N(C)Cc2ccccc2)c1Cl] | <1.0 |
| 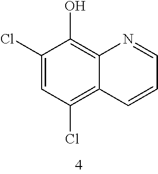 [SMILES: Oc1c(Cl)cc(Cl)c2cccnc12] | <1.0 |
| 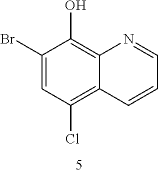 [SMILES: Oc1c(Br)cc(Cl)c2cccnc12] | <5.0 |
| 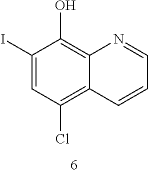 [SMILES: Oc1c(I)cc(Cl)c2cccnc12] | <10.0 |
| 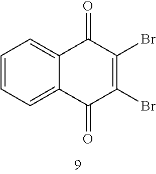 [SMILES: O=C1C(Br)=C(Br)C(=O)c2ccccc21] | <25.0 |
| 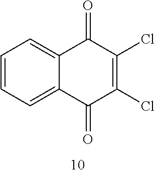 [SMILES: O=C1C(Cl)=C(Cl)C(=O)c2ccccc21] | <10.0 |

### Mycobacteria/HIV Co-Infections

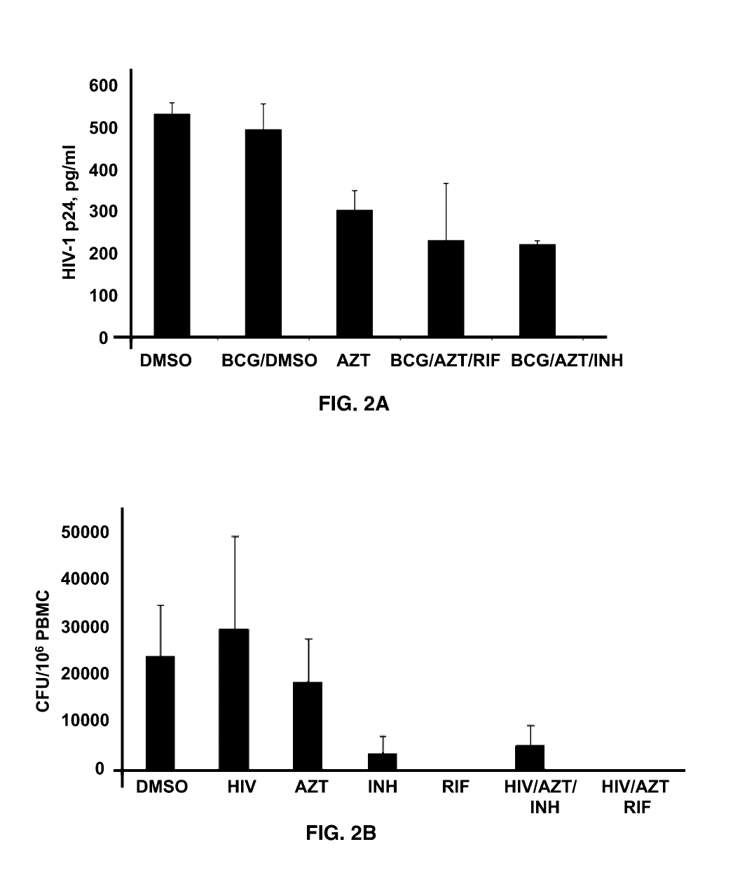

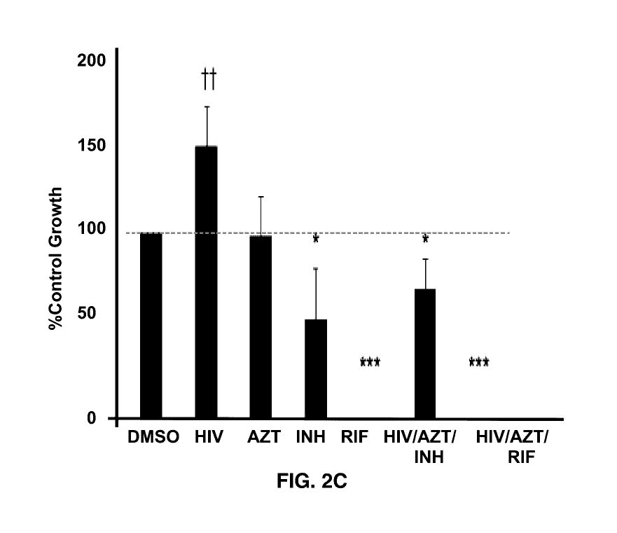

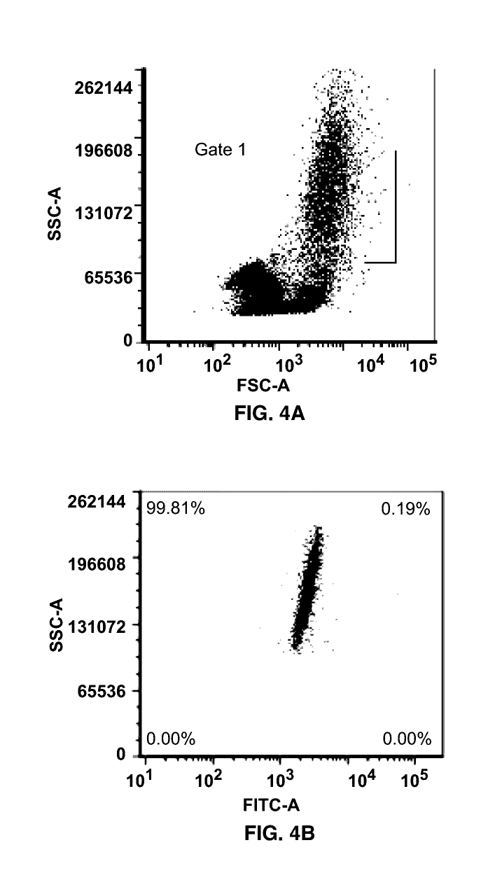

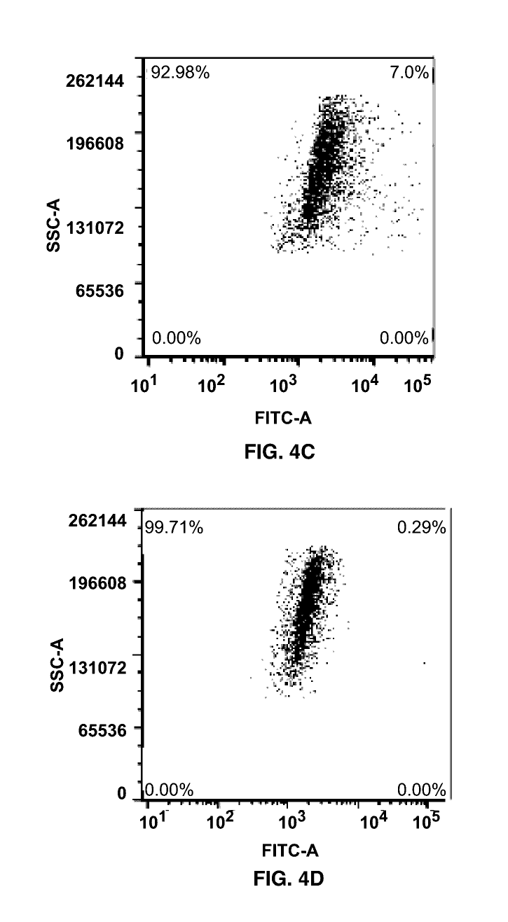

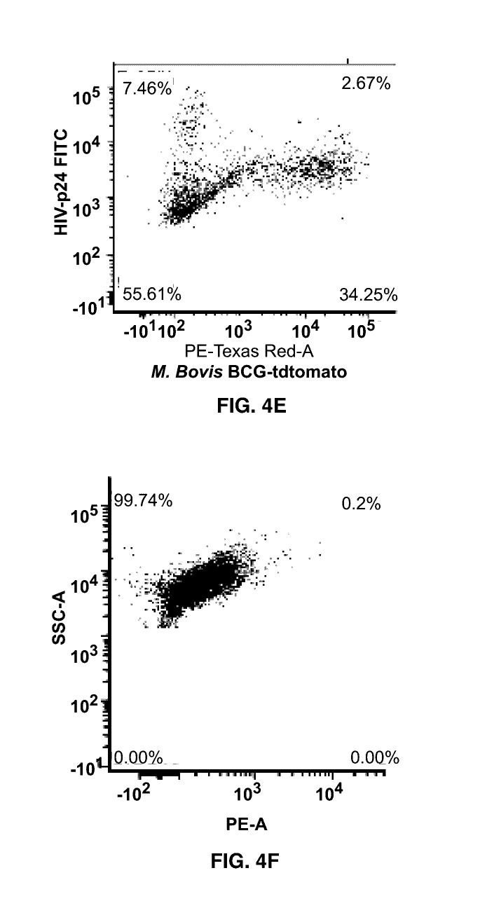

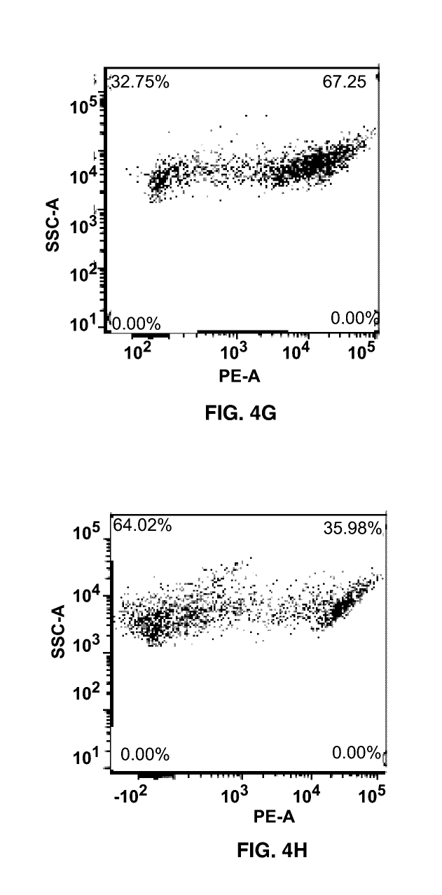

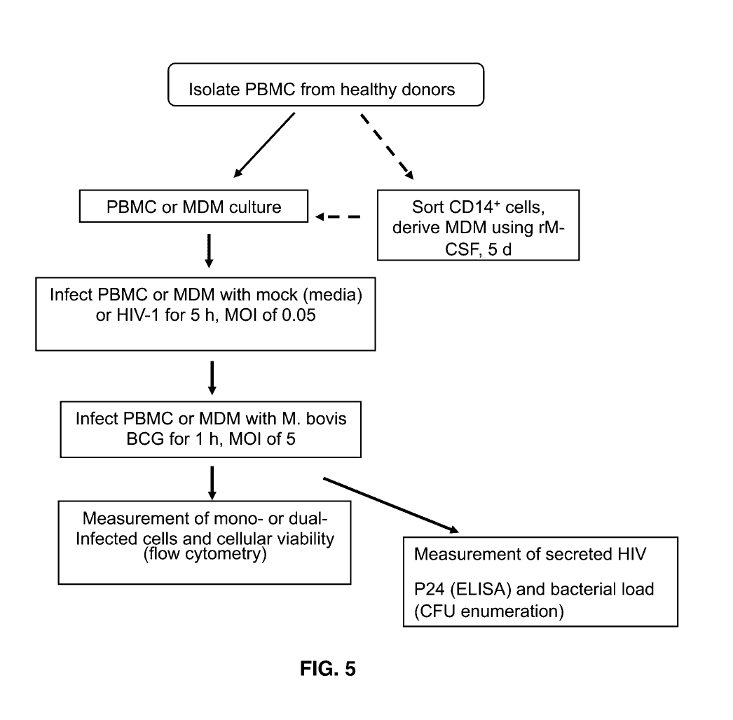

An in vitro co-infection model for application to discovery of novel compounds for treatment of TB was developed. As outlined in FIG. 5, PBMC were infected with a dual tropic strain of HIV-1 (89.6) for 5 h using a T-cell tropic HIV isolate (strain 213) to result in intracellular HIV infection. ELISA kit was used to characterize productive infection with HIV as the secretion of HIV-1 p24 protein into the culture supernatant is observed (FIG. 2A), while no p24 could be detected in mock-infected cultures (data not shown). The level of HIV p24 expressed by infected cultures is within the range expected in human biological samples and this measurable replication represented a fairly low number of virus-infected cells (FIG. 4A).

As shown in FIG. 2A, AZT reduces p24 expression in HIV infected cells compared to cultures treated with the carrier (DMSO) as a control. Co-infection of PBMC with BCG did not impact HIV replication as indicated by p24 secretion in both carrier- and AZT-treated cultures. However, antimycobacterial compounds Isoniazid (INH) and Rifamycin (RIF) did markedly alter AZT anti-viral activity as expected with regard to RIF because Rifamycin family member interactions with ARV require changes in liver enzyme levels in vivo.

Intracellular infection with M. bovis BCG in PBMC cultures exposed to live mycobacterium was further studied (FIG. 2B). Cultures were treated with gentamycin (50 μg/ml) to eliminate extracellular bacteria and the drug effectiveness against intracellular infection was determined. Treatment with standard anti-TB drugs (INH, RIF) dramatically reduced CFU from cellular lysates 7 d p.i. (FIG. 2B). To normalize the data across multiple donors, the CFU numbers of non-treated, non-HIV-infected cultures were set to 100% and the data was re-analyzed. As shown in FIGS. 2B-2C, HIV infection altered mycobacterial growth and potentially TB drug effectiveness in this model as an increase in intracellular M. bovis BCG replication due to co-infection of cultures with HIV-1. Co-infection with HIV increased mycobacterial growth on average to 150% of the control growth (FIG. 2C) while treatment with AZT reduced the viral load brought mycobacterial growth back to levels observed in the absence of HIV infection.

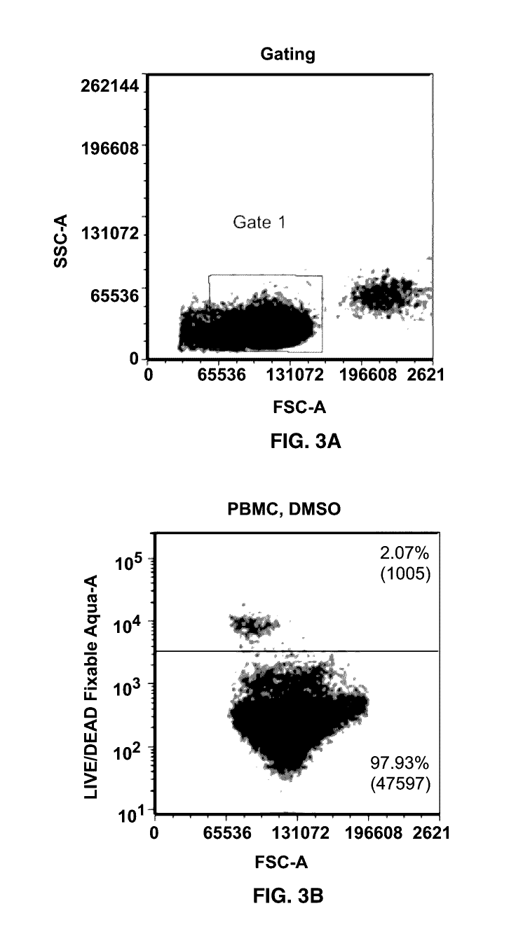

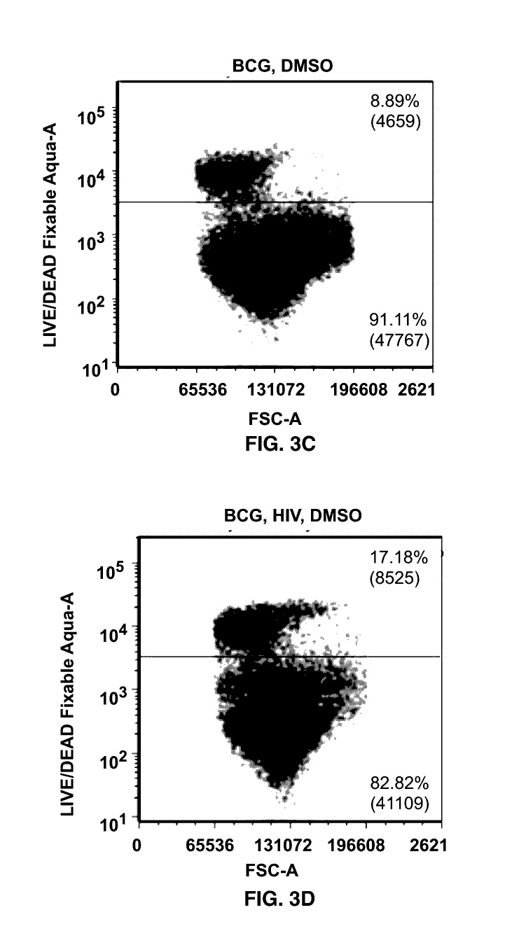

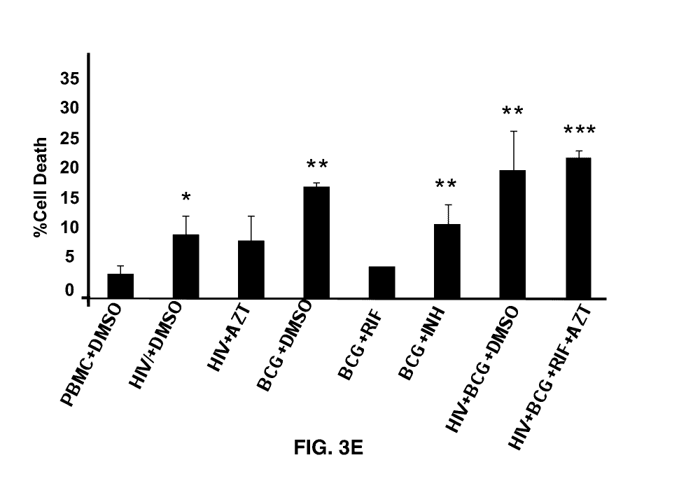

In order to perform high throughput analysis of various compounds by using the model developed herein, the screening assays were optimized using commercially available cell viability reagent (Invitrogen, fixable aqua live/dead viability marker) as shown in FIGS. 3A-3B. In contrast to many other cell viability determinations, this approach is not compromised by the formaldehyde fixation procedures required for safe flow cytometry analysis and the differences in cell viability due to mono- or dual-infection are easily visualized (FIG. 3A).

In samples from 2 donors, cellular viability using the standard tyrpan blue exclusion test was additionally determined. M. bovis BCG infection promotes greater cell death in cultures and co-infection with HIV exacerbates this effect in some donors. The toxicity of various compounds in addition to pathogen cytotoxicity can be further determined as demonstrated with standard TB and HIV drug. These toxicity and drug interactions issues are high priority considerations in TB/HIV endemic areas (Mcllleron, et al. 2007).

The live/dead marker shown in FIGS. 3A-3E were measured in one channel as part of a multi-color flow cytometry assay within a single tube or well in a 96-384 well plate. Additional fluorescent channels could then be incorporated to measure changes in pathogen load and other cellular indicators as desired for flow cytometric or bead array-based analysis. An example of using fluorescence-based detection of HIV and M. bovis BCG in co-infected cultures by flow cytometry was outlined in FIGS. 4A-4H. The data shown in FIGS. 4E-4H were obtained using a red (tdtomato) fluorescent M. bovis BCG construct and a FITC-labeled antibody to HIV-1 p24.

As shown in FIGS. 4A-4D, intracellular infection with HIV (7.5%), BCG (34%) and both (2.7%) were detected following 7 days of culture. The data demonstrates that most cells are mono-infected, though as shown in FIGS. 2A-2C, there were likely biological effects mediated indirectly that can impact mycobacterial growth. FIGS. 4E-4H further demonstrated the potential to monitor specific cellular populations as impacted by drug compounds or other interventions. Reduction of intracellular HIV and BCG was clearly observed following treatment with AZT and RIF, respectively. Optimization of this flow cytometric approach would be very amenable to high throughput screening of large compound libraries, as validate by ELISA and CFU enumeration.

### Example 4

### Cloning of LmMetAP1 in p1RIHYG Expression Vector

The LmMetAP1 gene was amplified from L. major genomic DNA using the oligonucleotides LmMetAP1-Xbal sense (5′-TCTAGAGGATCCATGCCCTGCGA AGGCTGCGGC-3′; SEQ ID NO: 1) and LmMetAP1-Xbal antisense (5′-TCTAGAGAATTCTCAGATTTTGATTTCGCTGGGGTCTTCGG-3′; SEQ ID NO: 2) primers. Polymerase chain reaction (PCR) was performed using PCR master mix (Promega, Madison, Wis.), 420 ng of L. major genomic DNA and LmMetAP1-sense and -antisense primers under denaturation of 5 minutes at 95° C., followed by 40 cycles of 60 seconds at 95° C., 60 seconds at 68° C., and 90 seconds at 72° C.; a final 5 minute elongation period at 72° C. was performed. The PCR product was ran in a 0.8% agarose gel and purified using Wizard SV Gel and PCR Clean-Up System (Promega, Madison, Wis.). The amplified LmMetAp1 gene was then cloned into p1 RIHYG expression vector by first digesting p1 RIHYG expression vector with Xba I restriction enzyme and then ligating the amplified LmMetAp1 gene and transformed into DH5a E. coli. Colonies were grown in 5 mL of Terrific Broth (TB) liquid media for 18 hours and plasmid DNA from bacterial pellets was collected using QIAprep Spin Miniprep Kit (Qiagen, Valenica, Calif.). The clones were sequenced (by the DNA Analysis Core Facility, Border Biomedical Research Center, El Paso, Tex.) and confirmed using Basic Local Alignment Search Tool (BLAST) and Multiple Sequence Alignment by CLUSTALW.

### Trypanosomatid Culture

Promastigote forms of L. major strain Friedlin clone V1 expressing firefly luciferase were grown in M199 medium supplemented with hemin and 10% fetal bovine serum (FBS) inactivated at 56° C. for 30 minutes and treated with 50 ng/mL streptothricin neosulfate for maintenance of LUC gene.

Leishmania major Luciferase Assay

Compounds were screened against L. major strain friedlin clone V1 promastigotes expressing firefly luciferase at concentrations ranging from 100 μM-780 nM in a 96 well plate. These experiments were done in triplicates using L. major media and 10⁶ parasites per well. The plates were incubated for 24, 48, 72 and 96 hours at 28° C. Amphotericin B (AB) was used as a positive control at 5 μM as a comparison to the leishmanicidal activity of the inhibitors. The inhibitory effects of the compounds were assessed by monitoring parasite survival through luciferase activity. The substrate 5′-fluoroluciferin (ONE-Glo Luciferase Assay System, Promega, Madison, Wis.) was added according to the manufacturer's protocol after each time point. The plates were read using a luminometer (Luminoskan, Thermo Scientific, Rockford, Ill.). Data was analyzed using GraphPad Prism Software (Graph Pad Software, Inc., La Jolla, Calif.). The compounds that showed >90% inhibition were chosen as “hits.”

### Mammalian Cell Lines Alamar Blue Assay

Inhibitors that produced effective antiparasitic activity in L. major were further tested in the mammalian cell lines RAW 264.7 murine macrophages, U2O5 human osteoblasts as well as in intraperitoneal mouse macrophages. For the cell lines, cells were plated as 10⁵ cells per well in a 96 well plate where previously diluted compounds ranging in concentrations from low nM up to 20 μM were transferred. The cells were incubated for 96 hours at 37° C. Intraperitoneal mouse macrophages were plated at a density of 10⁶ cells per well in a 96 well plate and were incubated for 8 hours at 37° C. Compounds were diluted in a separate 96 well plate in triplicate and then transferred to the plate containing the murine intraperitoneal macrophages. Together, the compounds and cells were incubated for 24 hours.

Mammalian cell cytotoxicity of the compounds was analyzed by monitoring the reduction of the growth medium serving as an indicator of metabolic activity. After each time point, AlamarBlue reagent (Invitrogen, Carlsbad, Calif.) was added according to the manufacturer's protocol. The plates were read using a fluorometer (Flouroskan, Thermo Scientific, Rockford, Ill.). Data was analyzed using GraphPad Prism Software (GraphPad Software, Inc., La Jolla, Calif.).

### Example 5

### Evaluation of the Anti-Leishmanial Activity of MetAP Inhibitors

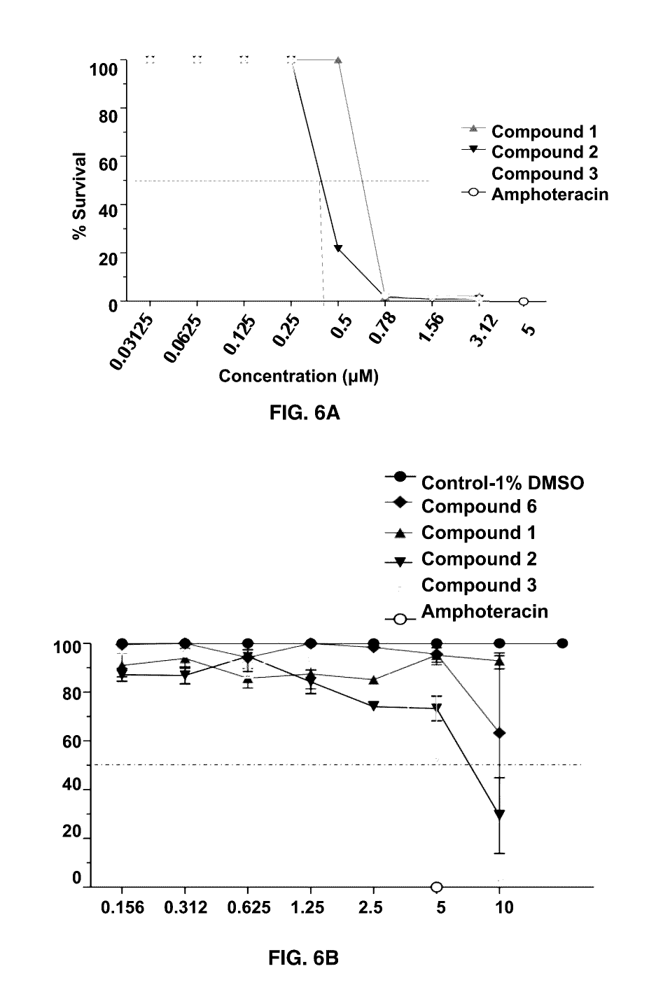

A chemical genetic approach was used to elucidate the function and relevance of methionine aminopeptidase 1 in Leishmania major. The study was begun using a whole cell based approach and three pharmacophores were identified as methionine aminopeptidase inhibitors (FIGS. 6A-6B). The effective concentration dose at 50% for compounds 1-3 was determined and are found to be in the lower micromolar range (Table 4). These inhibitors at the lower concentration of 0.78 μM are showing only 5-6% survival of L. major promastigotes (FIG. 6B). Particularly, compound 2 found to be the most specific for the LmMetAP1 and only a small amount of drug was necessary for the inhibition of LmMetAP1 (Table 4).

In order to confirm that the inhibitors were targeting LmMetAP1, a genetic approach was investigated. Overexpression of LmMetAP1 in vivo would cause resistance to the inhibitors if indeed the target was methionine aminopeptidase 1 (FIG. 6B). LmMetAP1 was cloned in the Leishmania expression vector pXG, and parasites were transfected. The parasites overexpressing LmMetAP1 were selected for and cloned. After addition of drug/controls (DMSO and AB), growth of promastigotes were monitored for 96 hours and development of resistance to the compounds was seen based on the EC₅₀ values being greater than 5 fold in the transgenic parasite than its wild type counterpart (Table 4). After cloning, overexpressing and purifying the protein to near homogeneity, the chromogenic substrate l-methionine-p-nitroanalide (I-Met-pNA) (Sigma) was used to determine inhibition of the enzyme using various compounds (Mitra et al. 2006). As described above, compound 2 found to be the most specific for the LmMetAP1 as seen by small amount of drug necessary for the inhibition of LmMetAP1.

TABLE 4
Evaluation of the Anti-leishmanial Activity of MetAP Inhibitors

| | | | L. major |
|---|---|---|---|
| | | Wild-type | promastigotes |
| | | L. major | over-expressing |
| | MetAP IC₅₀ | promastigotes | LmMetAP1 EC₅₀ |
| Compound | (μM) | EC₅₀ (μM) | (μM) |
| 1 | LmMetAP1: 10-100 | 0.64 | >10 |
| | HsMetAP1: 315.5 | | |
| | HsMetAP2: 145.6 | | |
| 2 | LmMetAP1: >2.5 | 0.375 | 9 |
| 3 | LmMetAP1: >1 | 0.243 | 5 |

### Example 6

### Sub-Cloning MetAP from E. faecalis

N-terminal poly-His-tag EfMAP1 was amplified by PCR from E. faecalis genomic DNA using Taq polymerase. The primers used were 5′-GCG GGA TCC ATT ACA TTA AAA TCA CCA C-3′ (SEQ ID NO: 3) and 5′-GCG CTC GAG TTA ATA AGT CAA TTC TC-3′ (SEQ ID NO: 4) for forward and reverse direction respectively. The PCR product was cloned into pET28a, using the BamH I and Xho I restriction sites respectively. The EfMetAP1 clone was verified by sequencing.

Over-Expression and Purification of Recombinant MetAP1 from E. faecalis

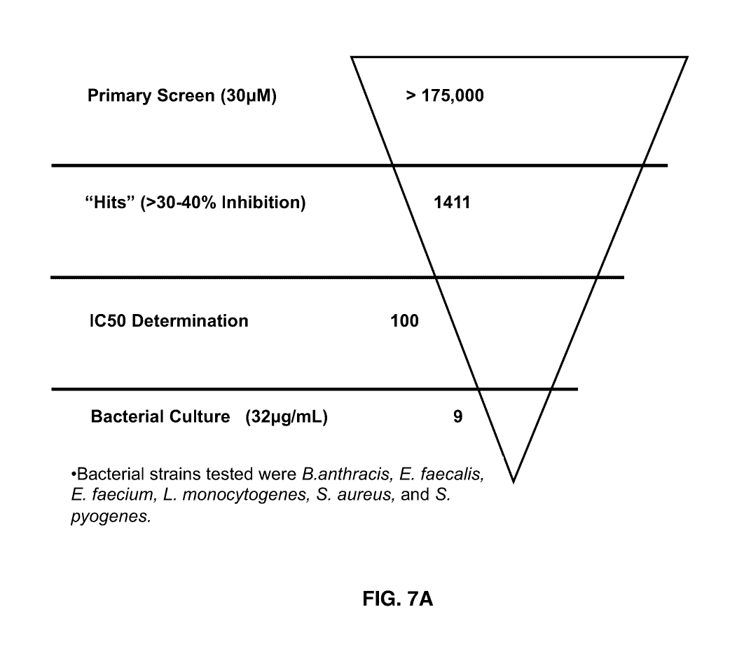

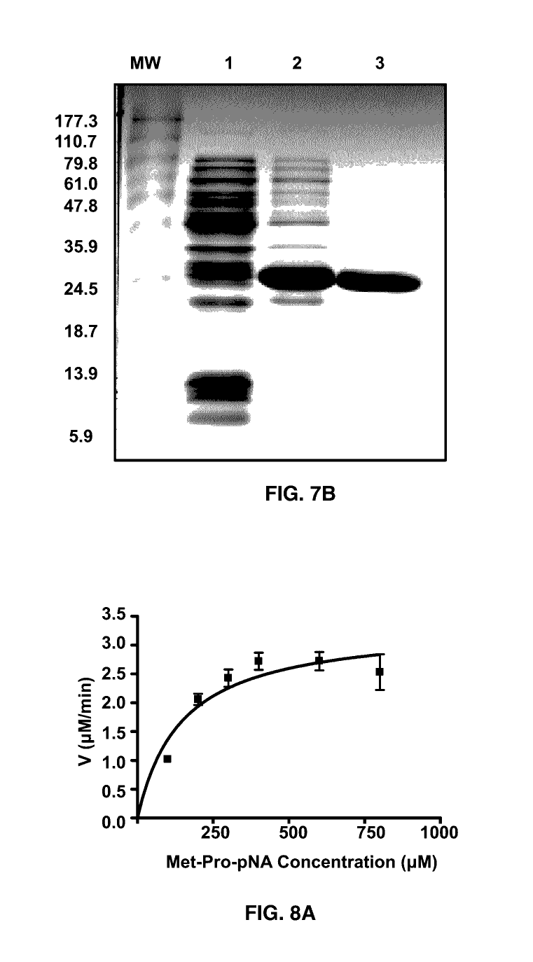

E. coli cells (BL21) containing the expression plasmid were cultured at 37° C. in 1 Liter of Listeria Broth (LB) containing 30 mg kanamycin until OD600 reached about 0.6-0.7. Thereafter, expression was induced by addition of isopropyl β-D-galactoside (IPTG) to a final concentration of 1 mM vibrating at 37° C., and 275 rpm for 4 hrs. The cells were harvested and washed with 1×PBS (137 mM NaCl, 2.7 mM KCl, 4.3 Na₂HPO₄.7H₂O, 1.4 mM KH₂PO₄). The cells were sonicated 4 times for 12 s in 1×PBS with 0.2% Triton-X-100 and EDTA-free Protease Inhibitor tablets. The resulting mixture was centrifuged at 8000 g for 10 mins. The supernatant was loaded unto pre-equilibrated (1×PBS) Talon beads. After binding for 30 min the beads were washed three times with basic buffer (10 mM Hepes pH 8.0, 100 mM KCl, 1.5 mM MgCl₂, 10% glycerol). The enzyme was eluted with 75 mM Imidazole in Basic buffer. The protein was quantified using the Bradford assay. The average yield for EfMetAP1 was 38.63 mg/L of culture respectively (FIG. 7B).

Biochemical Characterization of MetAP from E. faecalis

### Determination of Kinetic Constants

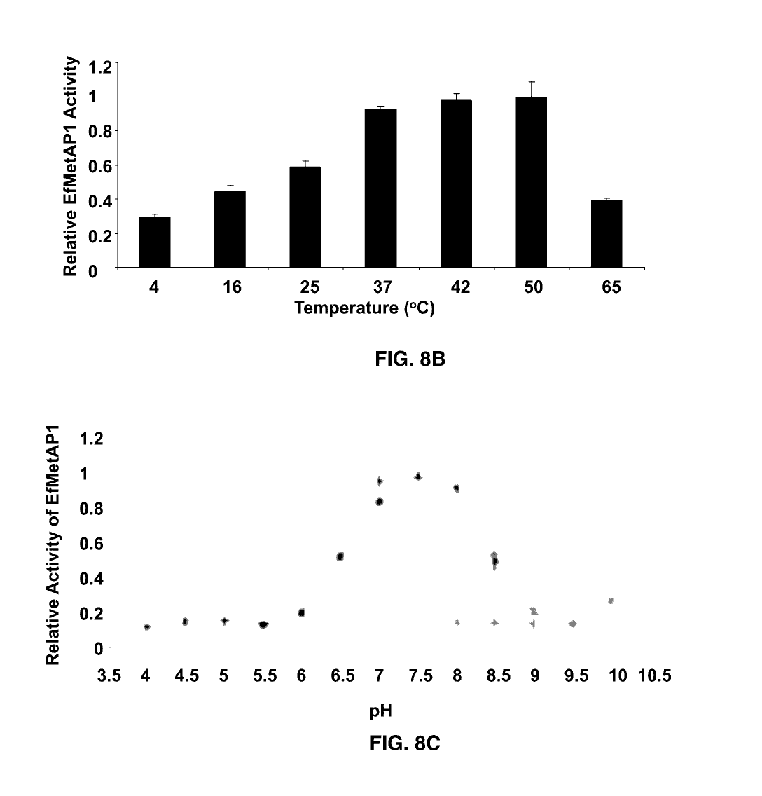

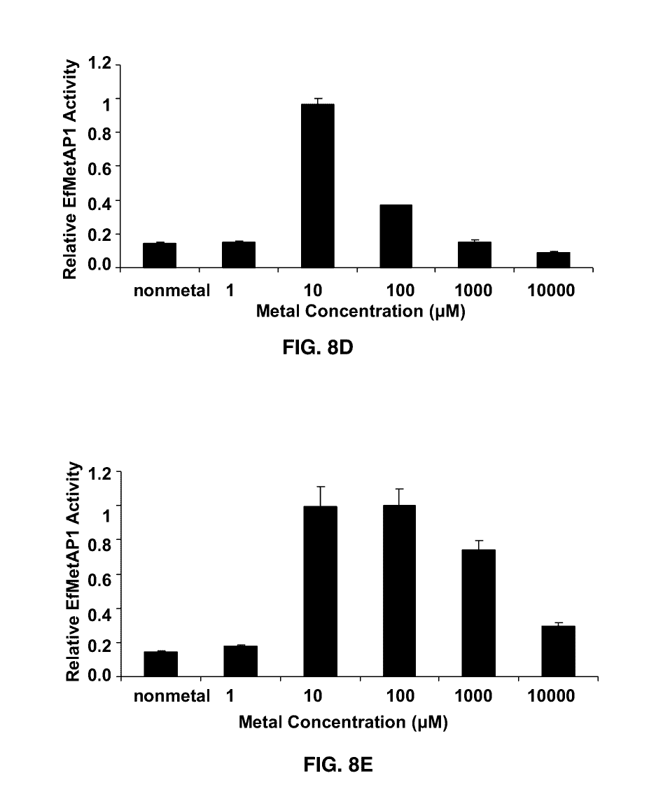

The kinetic constants of recombinant EfMetAP1 was determined using a coupled methionine-proline aminopeptidase assay (Zhou et al., Anal Biochem. 280(1):159-65, 2000). The substrate used in this assay is a dipeptide, Methione-Proline coupled to p-Nitroaniline. The kinetic constants were obtained by varying substrate concentrations from 0 μM to 800 μM measuring the resulting enzyme activity. The reactions were carried out in 96-well plates at room temperature and monitored at 405 nm on a spectrophotometer. The total reaction volume was 50 μL and each reaction contained 40 mM Hepes buffer (pH 7.5), 100 mM NaCl, 10 μM CoCl2, 100 μg/mL BSA, 0.1 U/mL ProAP, 0-800 μM substrate (Met-Pro-pNA), and 2.4 μM EfMetAP1 respectively. The background hydrolysis was corrected and the data was fitted against the Michealis-Menten equation: V=Vmax×[S]/(Km+[S]) using the Graphpad prism software for one-site binding hyperbola (FIG. 8A).

### Optimal Temperature

EfMetAP1 activity was determined at different temperatures from 4° C. to 65° C. (FIG. 8B). The total reaction volume was 50 μL and each reaction contained 40 mM Hepes buffer (pH 7.5), 100 mM NaCl, 100 μg/mL BSA, 0.1 U/mL ProAP, 600 μM substrate (Met-Pro-pNA), and 2.45 μM EfMetAP1. The reaction was allowed to go for 30 min, and then cooled to room temperature. Thereafter, ProAP was added and the reaction was monitored at 405 nm on a spectrophotometer. The background hydrolysis was corrected and the activities were determined relative to the optimal temperature.

### pH Dependence

The reactions were carried out in 96-well plates at room temperature by measuring the activities of recombinant EfMetAP1 using different buffers. The total reaction volume was 50 μL and each reaction contained buffer (50 mM sodium acetate pH 4.0-5.5, 50 mM MES pH 5.5-7.0, 50 mM HEPES pH 7.0-8.5, 50 mM Tricine pH 8.0-9.0 and 50 mM Ethanolamine pH 8.5-10.0), 10 mM NaCl, 1 μM CoCl₂, 100 μg/mL BSA, 0.1 U/mL ProAP, 600 μM substrate (Met-Pro-pNA), and 2.38 μM EfMetAP1 (FIG. 8C). The reaction was allowed to go for 30 min at room temperature. Then the MetAP reaction was terminated with 1 μL of 10% TFA, and neutralized with 1.4 μL of 1M NaOH. The pH was adjusted to 8.0 by addition of 5 μL of 1M HEPES buffer. Thereafter, ProAP was added and the reaction was monitored at 405 nm on a spectrophotometer. The background hydrolysis was corrected and the activities were determined relative to the optimal pH.

### Metal Dependence

After purification, recombinant EfMetAP1 was dialyzed into buffer C (50 mM Hepes buffer pH 7.0, 10 mM NaCl and 5 mM EDTA) at 4° C. overnight and the buffer was exchanged to buffer D (50 mM Hepes buffer (pH 7.0), and 10 mM NaCl) at 4° C. The metal dependence of EfMetAP1 was determined by measuring enzymatic activity in the presence of 1 μM-10 mM CoCl₂ (FIG. 8D) and MnCl₂ (FIG. 8E), using the methione-proline aminopeptidase assay. The reactions were carried out in 96-well plates at room temperature and monitored at 405 nm on a spectrophotometer. The total reaction volume was 50 μL and each reaction contained 40 mM Hepes buffer (pH 7.5), 100 mM NaCl, 100 μg/mL BSA, 0.1 U/mL ProAP, 600 μM substrate (Met-Pro-pNA), and 517 nM EfMetAP1. The MetAP reaction was allowed to go at room temperature followed by addition of ProAP. The background hydrolysis was corrected and the activities were determined relative to the optimal metal concentration.

### MetAP Inhibition:

175,000 compounds were screened against EfMetAP1, using the coupled methionine-proline aminopeptidase spectrophotometric assay (FIG. 7A). The primary screen was performed at final concentrations of 30 μM in 384-well plates using a titertek instrument with liquid handling capabilities coupled to a spectrophotometer. The compounds were dissolved in dimethylsulfoxide (DMSO) and the total reaction volume was 50 μL where each reaction contained 40 mM Hepes buffer (pH 7.5), 100 mM NaCl, 100 μg/mL BSA, 0.1 U/mL ProAP, 1.5 mM CoCl₂, 600 μM substrate (Met-Pro-pNA), and 1.38 μM EfMetAP1. The Enzyme was pre-incubated with compounds for 20 min at room temperature followed by addition of substrate. The reaction was incubated at room temperature for 30 min and monitored at 405 nm on a spectrophotometer. The compounds that showed greater than 30-40% inhibition were chosen as “hits”. It was determined the concentration needed for 50% inhibition in 96-well plates at final concentrations of 100 μM-300 nM. The background hydrolysis was corrected and the data was fitted against the sigmoidal-dose response (variable slope) equation using GraphPad prism software.

### Effect of EfMetAP Inhibitors on Clinically Relevant Pathogens

The effect of EfMetAP inhibitors against Enterococcus faecalis, Enteroccocuss faecium, Bacillus anthracis, Staphylococcus aureus, Listeria monocytogenes and Streptococcus pyogenes was determined. The primary screen was conducted with 102 compounds at 32 μg/mL. The compounds were dissolved to give a final concentration of 32 μg/mL in 17 mL molten agar, 2 mL compound solution (320 μL of 2 mg/mL compound in 1.68 mL water) and 1 mL sheep blood. The plates were poured at 45° C. and left overnight. The plates were inoculated with each bacterial strain in triplicates and allowed to dry at room temperature for 1 hr then incubated for 24 hr and scored respectively on the next day.

Assay of E. faecalis Inhibition

To determine the Minimum Inhibitory Concentration (MIC) of E. faecalis, bacterial culture strains OG1Rf, V583 and PMV 158 were grown in Brain Heart Infusion (BHI) culture by incubating overnight at 37° C. Initial Optical density (OD) reading was taken and culture was normalized to 1×106 cells; A600=0.6. Serial dilutions of the respective compounds were performed and 5 μl of each concentration was plated on to 96-well plates (Fisher brand, Becton Dickson Microtest tissue culture plate #353072) with 45 μl of BHI containing 5 μl of E. faecalis (three wells per condition). Initial reading is taken at OD600 using a SpectraMax Gemini XPS plate reader (Molecular Devices, Sunnyvale, Calif.). The plate was incubated for 24 hr at 37° C. after which the final reading was taken at OD600. Kaplan-Meier log rank analysis was used to compare survival curves pairwise. P values of <0.05 were considered to be statistically significant. The software GraphPad Prism (version 5.0) was used for the analyses.

Liquid-Based Assay Using C. elegans as an Animal Model of Infection

In order to determine the Minimum Protective Concentration (MPC), synchronized glp-4; sek-1 worms were obtained from gravid adults by incubating eggs overnight in M9 buffer at 25° C. The L1-stage nematodes were hatched and plated on lawns of Nematode Growth (NG) agar media containing E. coli. Nematodes were further incubated to grow to sterile L4 larvae which are washed and then transferred onto lawn of E. faecalis grown on BHI agar plates, containing 50 μg/mL gentamycin, incubated for 5 hr at 25° C. and re-suspended in M9 buffer. Control and infected worms were transferred without agitation to 6-well plates containing 80% M9 buffer and 20% BHI plus 20 μl of compound to give a total volume of 2 mL per well (30 worms per condition). The plates were incubated at 25° C. and worms were scored daily for survival by shaking the plate and observing under a surgical microscope for motion. Dead worms did not move and exhibited rigid muscle tone. Based on this experiment it was determined if the compounds were also toxic to the worms as well as the efficacy. Kaplan-Meier log rank analysis was used to compare survival curves pairwise. P values of <0.05 were considered to be statistically significant. The software GraphPad Prism (version 5.0) was used for the analyses.

Assessment of Bacterial Viability after Treatment In Vivo

Young Adult nematodes sequentially preinfected with E. faecalis for 5 h. The nematodes were washed three times with sterile M9W and collected by centrifugation between each wash and then transferred into tubes. The wash was repeated twice using 25 mM Tetramisole hydrochloride and the supernatant was removed leaving about 50 μl. 500 μl of 25 mM tetramisole hydrochloride, 1 mg/mL of ampicillin and kanamycin are added to the tube and nematodes were incubated for 1 h to kill off excess bacteria. The nematodes were washed twice in 25 mM Tetramisole and transferred to 6 well plates containing 20% BHI and the inhibitors to recover for 48 h and 72 h. After recovery time elapsed, nematodes were then transferred into an eppendorf tube containing 200 μl of M9 and ground using motorized pestle for 1 min and tube was briefly vortexed. Ground worms were diluted and plated on BHI agar containing 10 μg/mL of Gentamycin and incubated at 37° C. for 24 h after which CFUs were counted.

### Example 7

### Characterization of EfMetAP1

Characterization experiments using the enzyme assay conditions were carried out to determine the kinetic constant, metal dependence and the effects of temperature and pH on the enzyme. The kinetic constant for EfMetAP1 was determined by measuring enzyme activity at different substrate concentrations ranging from 0 to 800 mM (FIG. 8A; Table 5). The Km was determined to be 163±88 μM (Table 5). The temperature profile showed an increase in enzyme activity as the temperature was increased from 4° C. to 50° C. with optimal enzyme activity at 50° C. (FIG. 8B) before loss of activity was seen at 65° C. The pH profile was achieved by performing the assay in different pH buffers. EfMetAP1 shows a broad range of activity (7.0-8.0) with resulting optimal pH at pH 7.5 using 50 mM HEPES buffer (FIG. 8C).

The metal dependence of EfMetAP1 using Co²⁺ and Mn²⁺ as the cofactors was investigated. The metal screen was performed by varying the metal concentration used from 0 to 10,000 μM. EfMetAP1 showed a broad range of activity, from 10 to 100 μM, in Mn²⁺ with a slight decrease in relative activity at higher concentrations (FIG. 8E). In contrast, when Co²⁺ was used the enzyme showed maximum activity at 10 μM and concentration-dependent inhibition at higher concentrations (FIG. 8D).

TABLE 5
Kinetic constants for methionine aminopeptidase from E. faecalis
using a dipeptide substrate (Met-Pro-pNA)

| Kinetic Constants | EfMetAP1 |
|---|---|
| Km (μM) | 163 ± 88 |
| Kcat (s⁻¹) | 0.02 |
| Kcat/Km (M⁻¹min⁻¹) | 8.9 × 10³ |
| Vmax (μM/min) | 3.5 ± 0.8 |

### Identification of EfMetAP1 Inhibitors

Small molecule library of 175,000 compounds against EfMetAP1 at a final concentration of 30 μM in 384-well plates using a coupled enzymatic assay was screened and 7-bromo-5 chloroquinolin-8-ol 5 was identified as an inhibitor of EfMetAP1. In order to investigate the structure-activity relationships, other structural analogs are acquired (Table 6). It was discovered that substitutions to the ortho-bromine of compound 5 for 3-cyclohexyl-1,3-oxazinan-3-ium increased enzyme activity whereas substitution of the ortho-bromine position for iodine reduced the IC₅₀. A reduction in IC₅₀ was also observed when the hydroxyl group of compound 5 was substituted for an acetoxy group and the ortho-bromine substituted for chlorine. Among all inhibitors tested, compound 8 was found to be most potent against EfMetAP1 enzyme with IC₅₀ value of 5.24 μM (Table 6).

TABLE 6
Effects of 7-bromo-5-chloroquinolin-8-ol and its analogs
on EfMetAP1 enzyme activity

| | EfMetAP1 |
|---|---|
| Chemical Structure | IC₅₀ (μM) |
| 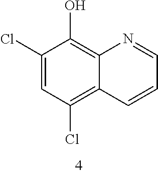 [SMILES: Oc1c(Cl)cc(Cl)c2cccnc12] | ND |
| 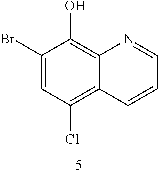 [SMILES: Oc1c(Br)cc(Cl)c2cccnc12] | 11.96 |
| 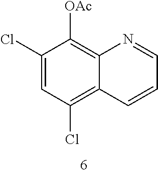 [SMILES: CC(=O)Oc1c(Cl)cc(Cl)c2cccnc12] | 14.63 |
| 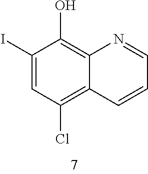 [SMILES: Oc1c(I)cc(Cl)c2cccnc12] | 18.41 |
| 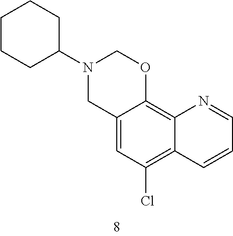 [SMILES: Clc1cc2c(c3ncccc13)OCN(C1CCCCC1)C2] | 5.24 |

Inhibition of E. faecalis Growth In Vitro by EfMetAP1 Inhibitors

Five inhibitors were tested for their effects on E. faecalis growth in culture to obtain the minimum inhibitory concentrations (MICs). Compounds 5 and 6 were more potent against the clinical OG1RF strain of E. faecalis, achieving MIC₃₀ between the ranges of 2.5-5.0 μg/mL, while the other three analogues had MIC₃₀ values between the ranges of 5.0-10.0 μg/mL. Compound 5, compound 6 and compound 7 achieved MIC₃₀ between the ranges of 0.25-0.5 μg/mL against the V583 strain, while compound 4 and compound 8 achieved MIC₃₀ between the ranges of 0.5-1.5 μg/mL. All inhibitors were between six to ten times more potent against the V583 than the OG1RF strain (Table 7).

TABLE 7
Effects of compounds quinolines 4-8 on E. faecalis

Minimum Inhibitory Concentration
(MIC₃₀, μg/mL)

| | | |
|---|---|---|
| Compound | OG1RF | V583 |

| | | |
|---|---|---|
| 4 | 5.0-10.0 | 0.50-1.50 |
| 5 | 2.5-5.0 | 0.25-0.50 |
| 7 | 5.0-10.0 | 0.25-0.50 |
| 8 | 5.0-10.0 | 0.50-1.50 |
| 6 | 2.5-5.0 | 0.25-0.50 |

### Example 7

### Assessment of Cytotoxicity and Efficacy of EfMetAP1 Inhibitors in a C. elegans Animal Model

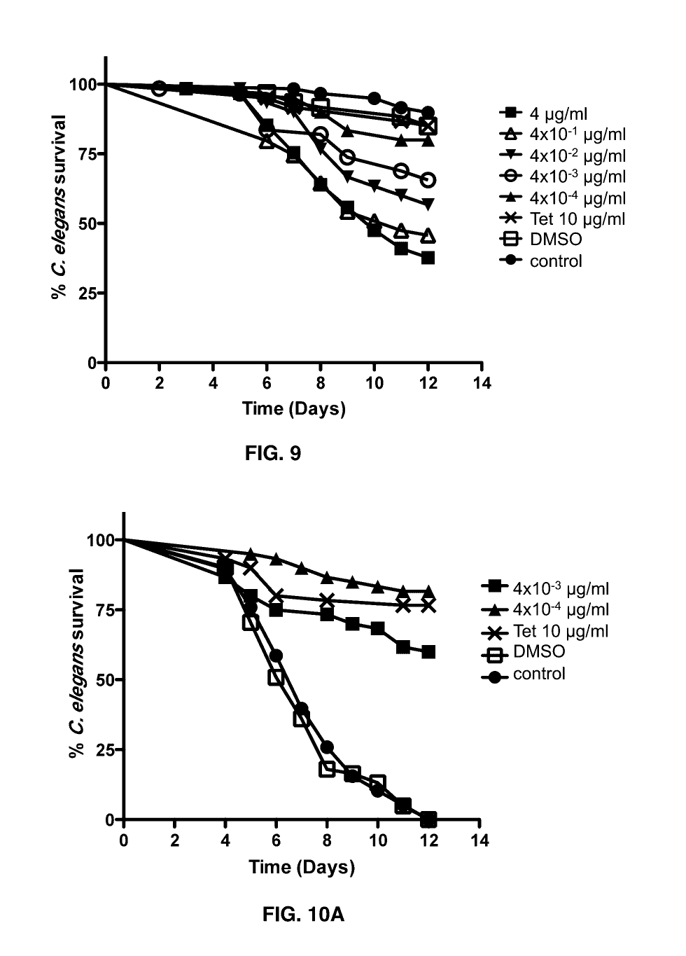

Toxicity and efficacy of EfMetAP1 inhibitors was assessed using C. elegans animal model. The toxicity of the inhibitors was first determined by exposing non-infected animals to different concentrations. Controls included the addition of 10 μg/mL of tetracycline, an antibiotic approved for human use at a concentration that is non-toxic and protective against infection in the worm model as a positive control and DMSO, the vehicle used to dissolve and deliver the EfMetAP1 inhibitors as a negative control. The controls did not decrease survival of the non-infected animals compared to no addition as shown in FIG. 9. Non-infected animals treated with concentrations ranging from 4×10⁻³ μg/mL to 4 μg/mL of compound 5 experienced some mortality up to approximately 40%, this was likely due to the toxic effects at this concentration.

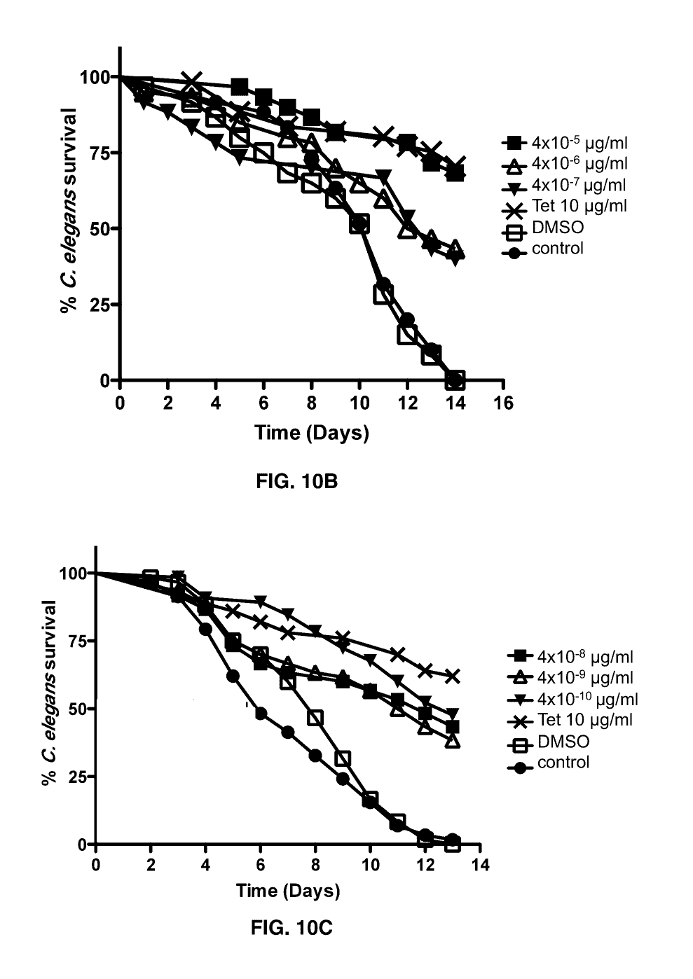

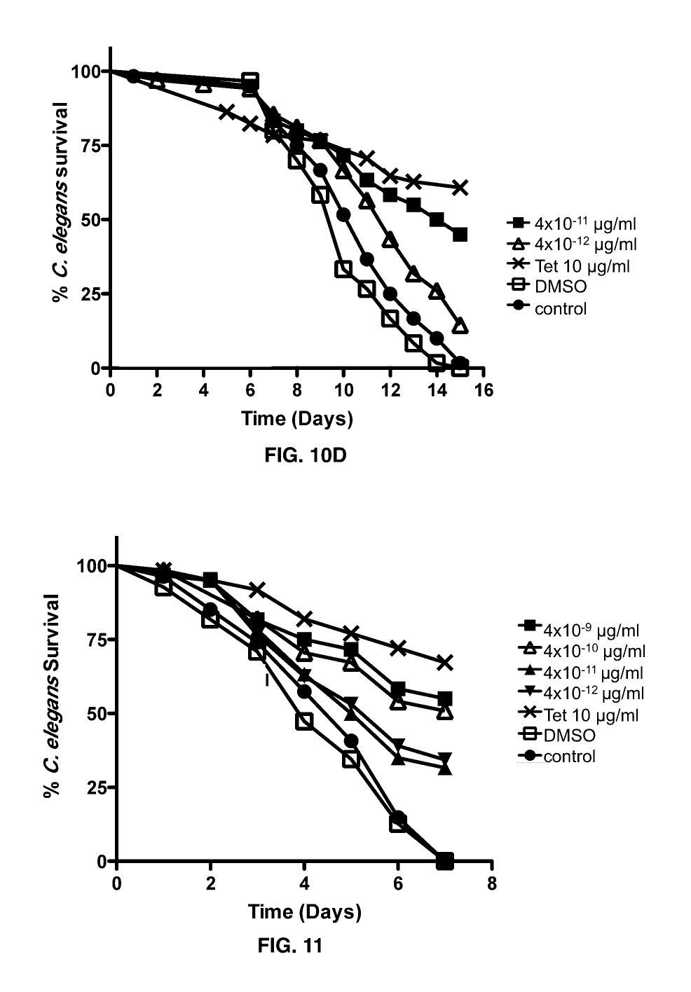

To test if non-toxic concentrations of the EfMetAP1 inhibitors were capable of protecting C. elegans from infection, animals infected with E. faecalis OG1RF are exposed to concentrations of inhibitors ranging from 4×10⁻¹² μg/mL to 32 μg/mL. Surprisingly, for compound 5 concentrations ranging from 4×10⁻⁵ μg/mL to 4×10⁻⁴ μg/mL the animals survived just as well, or better, than animals treated with tetracycline (FIGS. 10A-10B). In order to determine the concentration at which compounds 4-8 were no longer protective, lower and lower concentrations were tested by generating ten-fold dilutions. As observed in FIGS. 10A-10D, concentrations at 4×10⁻⁴ μg/mL and 4×10⁻⁵ μg/mL were the most protective and comparable to treatment with tetracycline. Though a decrease in protection was observed at lower concentrations, a significant loss of efficacy was not observed until a dilution of 4×10⁻¹² μg/mL was reached. The minimal protective concentration (MPC) of compound 5 was calculated to be 4×10¹¹ μg/mL. (FIG. 10 D) and Maximum protective concentration was determined to be 4×10⁻⁴ μg/mL (FIG. 10A) yielding approximately 80% survival of the nematodes. Compounds 4-8 had MPCs ranging from 4×10⁻¹² μg/mL to 4×10⁻⁹ μg/mL (Table 9A).

The effect of compound 5 and its analogues on animals infected with V583 was also examined. As shown in FIG. 11 and Table 9B, very low concentrations of these compounds were also protective and some protection was still observable at a concentration of 4×10⁻¹² μg/mL. Overall, these results demonstrate that the MPC of this class of inhibitors is many-folds lower than the MIC determined to prevent growth of E. faecalis cultures in vitro.

It is proposed that Compound 5 and its analogues are conferring protection at such low concentrations due to one or a combination of the following reasons: First, they may confer protection by activating stress response pathway that releases cytoprotective proteins. Studies have shown that C. elegans responds to oxidative stresses caused by xenobiotics and metal toxicants by activating the Antioxidant Response Element (ARE) via the Nrf2 pathway. The Nrf2 orthologue in C. elegans SKN-1 has been shown to induce expression of ARE by structural relative of the inhibitors; Napthoquinolones which resulted lifespan extension in C. elegans (Hunt=[59]). Interestingly CQ has been shown to be a zinc ionophore (Ding=[60]) and that sub cytotoxic levels of zinc are able to induce expression of HO-1 (Xue=[61]). HO-1 is a cyto protective enzyme that responds by decreasing oxidative stress, weakened inflammatory response and lower apoptosis rate (Ioboda=[62]).

Secondly by acting as an ionophore, the inhibitors are increasing the metal concentrations in the cell causing the activation of Metal Response Elements (MRE). Activation of MRE's by increases cellular levels of zinc has been shown to induce the expression of metalloprotease called Metallothionein which is important role in detoxification, homeostasis and protection from oxidative stress inflammatory responses and other stressors (Z-Ghandour, Vallee, Klassen=[63-65]). Schmeisser et al established that Mtl-2 is essential for lifespan extension in C. elegans that were exposed to low dose arsenite; a known toxicant (Schemiesser et al=[66]). Metallothioneins also plays a role in immune response to metal toxins, xenobiotics, oxidative stress caused by infections, inflammation, and physical stress as studies have demonstrated that MTs are responsible for lymphocyte, macrophage proliferation plus enhances the cells ability to kill phagocytized organisms (Lynes and Youn=[67-68]). White et al showed that in the presence of zinc, CQ increased efflux of zinc, which led to the up regulation of metalloproteinase activity (White=[69]) that could possibly be happening in or C. elegans assay.

Thirdly, low doses of the compounds may be transiently inducing the expression of ROS which also leads to activation of the SKN-1 signaling cascade (Hoeven and Garsin 2011=[70]) leading to the activation of the MRE and ARE protective responses. Schmeisser et al showed that low dose arsenite transiently induces a ROS and p38MAPK signaling pathway that leads to lifespan extension on C. elegans. Due to the promiscuous nature of the inhibitors it is believed that they can have multiple targets in vivo. The increase in potency of the inhibitors in the C. elegans is attributed to the possibility that the inhibitors are binding to other receptors that activate the immune system as well as triggers the expression of genes that regulate other defensive mechanisms in the nematode that help clear the E. faecalis infection.

Specifically it is hypothesized that the inhibitors are increasing the expression metallothioneins (especially CeMT2) in the nematodes gut; which has known protective effects on the nematodes. Metallothioneins (MTs) are a family of small, highly conserved small cysteine rich metalloproteases. They are important in metal detoxification, homeostasis and protection from oxidative stress. Studies have shown that extracellular MT causes a burst in superoxide in peritoneal macrophages and this increase in metabolic activity leads to increase in the cells ability to kill phagocytized yeast. Intracellular MT contributes to increase in monocytes activation by while reduction of MT may limit efficacy. MTs promotes murine Lymphocyte proliferation. In C. elegans they scavenge and protect against reactive oxygen species and oxidative stress with CeMT-2 being more effective than CeMT-1. mtl-2 as well as a mitochondrial transporter tin-9 are both essential for lifespan extension of nematodes exposed to low doses of Arsenite. Therefore the protections seen in the nematode are likely due to the combination of the many advantageous effect of metallothionein.

TABLE 8
Effects of EfMetAP inhibitors on C. elegans infected with
OG1RF and V583

| | OG1RF | | V583 | |
|---|---|---|---|---|
| | MmaxPC | MPC | MmaxPC | MPC |
| Inhibitor ID | (μg/ml) | (μg/ml) | (μg/ml) | (μg/ml) |
| 4 | 4 × 10⁻⁸ | 4 × 10⁻¹¹ | ND | 4 × 10⁻¹² |
| 5 | 4 × 10⁻⁴ | 4 × 10⁻¹¹ | ND | 4 × 10⁻¹² |
| 6 | 4 × 10⁻⁹ | 4 × 10⁻¹² | ND | 4 × 10⁻¹¹ |
| 7 | 4 × 10⁻¹² | 4 × 10⁻⁹ | ND | 4 × 10⁻¹² |
| 8 | 4 × 10⁻⁸ | 4 × 10⁻¹¹ | ND | 4 × 10⁻¹¹ |

ND: Not Determined,
MPC: Minimum Protective Concentration,
MmaxPC: Maximum Protective Concentration

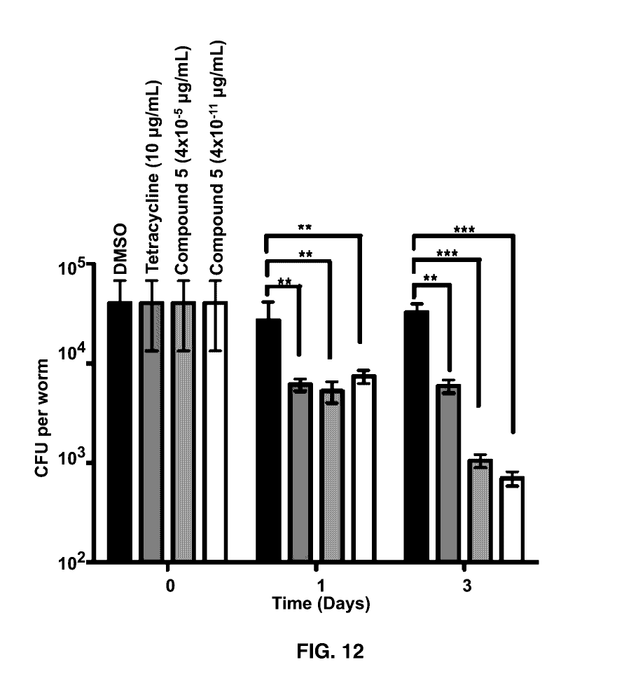

The ability of the compound 5 to reduce colonization of the intestinal tract was examined. As shown in FIG. 12, about 10⁵ CFU/worm were observed following exposure to E. faecalis OG1RF for 24 hours. When the animals were treated with the Minimum Protective Concentration (MPC) of compound 5 which is 4×10⁻¹¹ μg/mL, a 50-fold drop in CFU after three days of treatment was observed (FIG. 12). These data indicates that exposure to the compound resulted in a significant drop in bacterial colonization, which correlated with the increased survival (FIGS. 10A-10D).

### Example 8

### Assessment of the Efficacy EfMetAP1 Inhibitors in Other Clinically Relevant HAI Causing Pathogens

An assessment of the efficacy of the inhibitors on other clinically relevant pathogens that are responsible for nosocomial infections, as well as to predict the efficacy of the compounds in treatment of polymicrobial infections was performed. Last CDC estimates report that 16.4% of HAI were polymicrobial and one of the leading causes of gram-negative infections is Pseudomonas areuginosa. P. areuginosa is a gram-negative bacteria that causes infections in healthcare settings the most serious of which include: bacteremia, pneumonia, urosepsis and wound infections including secondary infection of burns. P. areuginosa was ranked 5^{th} among the causative pathogens of HAI in the US with 7.5% of infections. Most importantly, infection rates are reported to be rising both in the US and globally as well as resistance to antibiotics. P. areuginosa is resistant to many antibiotics such as quinolones, carbapenems and Aminoglycosides. It has been dubbed an “ESKAPE” pathogen due to high incidence in HAI's and its ability to evade the activity of antibacterial drugs. These factors make P. areuginosa an important pathogen for which development of new therapeutics is imperative.

Screening of Select EfMetAP1 Inhibitors Against P. aeruginosa

P. aeruginosa bacterial culture strain PA14 was grown in LB media and SK media by incubating overnight at 37° C. (Rahme et al. 1995). Initial Optical density (OD) reading was taken and culture was normalized to 1×106 cells; A600=0.6. Serial dilutions of compounds 4 and 5 were performed to obtain final concentrations of 40 μg/ml to 2.5 μg/mL after 5 μl of each concentration was plated onto 96-well plates (Fisher brand, Becton Dickson Microtest tissue culture plate) with 45 μl of the respective media containing 5 μl of E. faecalis (three wells per condition). Initial reading is taken at OD600 using a MultiSkan MCC Plate reader plate reader (Fisher Scientific, Pittsburgh, Pa., USA). The plate was incubated for 24 hours at 37° C. after which the final reading was taken at OD600.

### Assessment of the Efficacy EfMetAP1 Inhibitors in Other Clinically Relevant HAI Causing Pathogens.

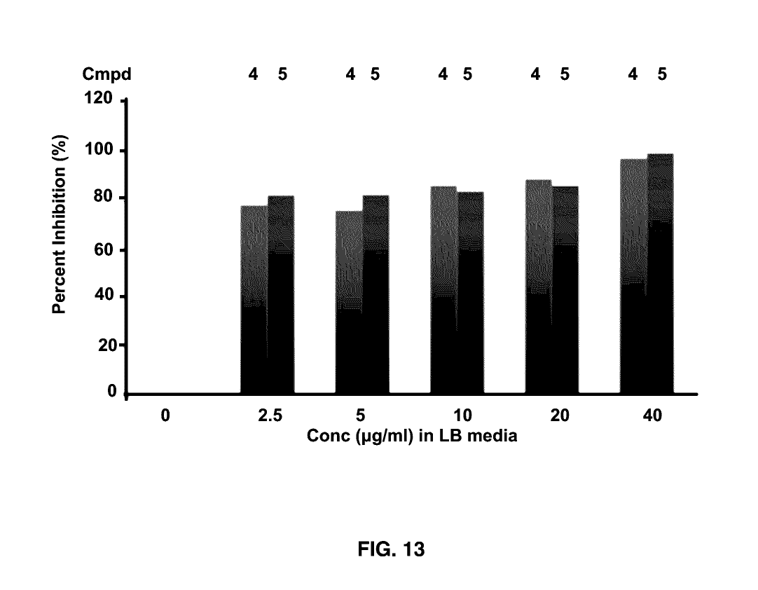

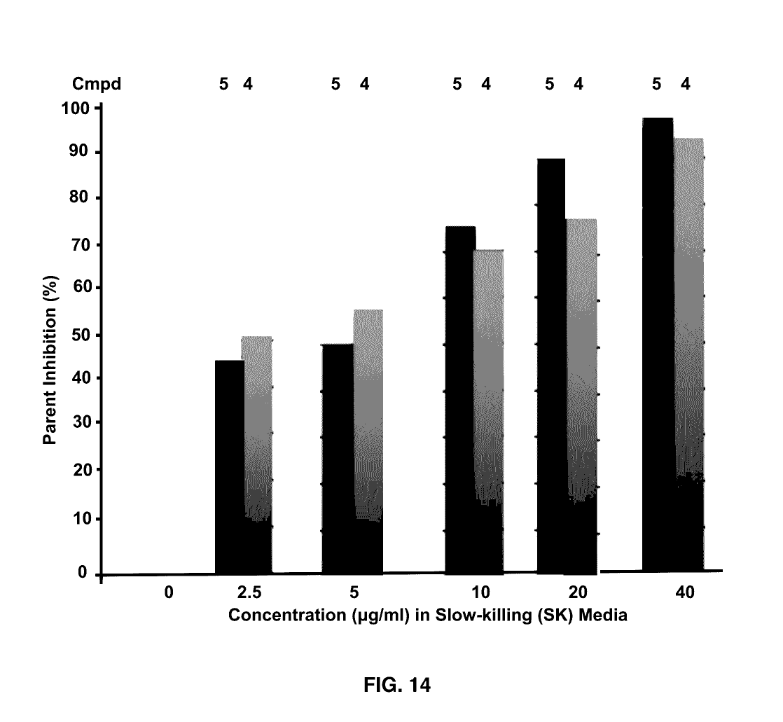

The preliminary screening of compounds 4 and 5 against P. aeruginosa infected C. elegans was performed according to standardized assay developed by Rahme et al. 1995 with modifications. The result showed that in LB media the inhibitors were able to achieve approximately 80% inhibition at a concentration of 2.5 μg/mL (FIG. 13). In both media, the MIC₃₀ of inhibitors is <2.5 μg/mL (FIGS. 13-14). The inhibitors achieved a greater degree of inhibition against this clinically pathogen than in E. faecalis OG1RF and therefore are a potential candidate for use in polymicrobial nosocomial infections.

The ability of the inhibitors to not only inhibit growth of gram-positive E. faecalis but also be efficacious against gram-negative P. aeruginosa shows that this pharmacological class of compounds have a broad spectrum of activity. This is of clinical relevance as many nosocomial infections are polymicrobial. Therefore the use of a broad spectrum antibiotic that is potent would reduce the pill burden on patients, prevent drug-drug interactions and their associated cytotoxic effects and ultimately improve treatment outcomes.

What is claimed is:

1. A methionine aminopeptidase inhibitor, having a structure Formula I: 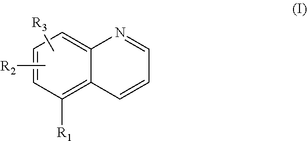
wherein
R₁ is a halogen; and
R₂ and R₃ independently are halogen, OH or —OC(O)CH₃, or R₂ and R₃ together form an N-substituted 1,3-oxazinanane; or
a pharmaceutically acceptable salt or a regioisomer thereof or a combination thereof.

2. The methionine aminopeptidase inhibitor of claim 1, wherein the chemical structure thereof is:

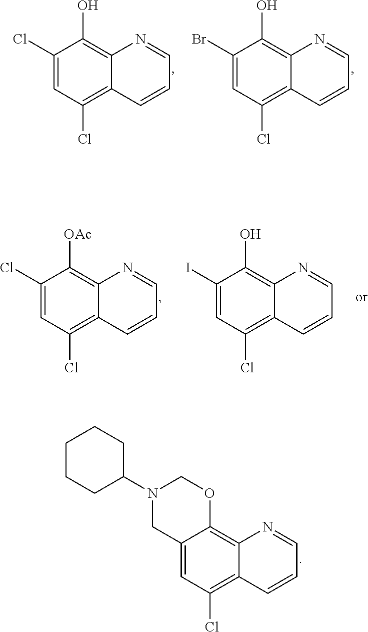

3. A methionine aminopeptidase inhibitor, having a structure Formula II: 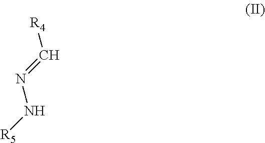
wherein
R₄ is 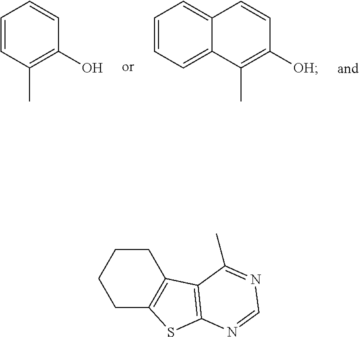
R₅ is isonicotonyl group or; or a pharmaceutically acceptable salt or a regioisomer thereof or a combination thereof.

4. The methionine aminopeptidase inhibitor of claim 3, wherein the chemical structure thereof is:

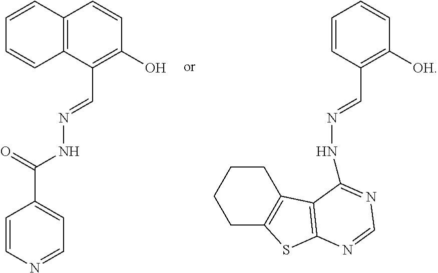 [SMILES: O=C(N/N=C/c1c(O)ccc2ccccc12)c1ccncc1 ; "or" ; Oc1ccccc1/C=N/Nc1ncnc2sc3c(c12)CCCC3]

5. A methionine aminopeptidase inhibitor, having a structure Formula III:
wherein X is a halogen.

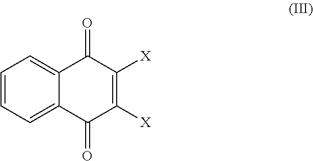

6. The methionine aminopeptidase inhibitor of claim 5, wherein the chemical structure thereof is:

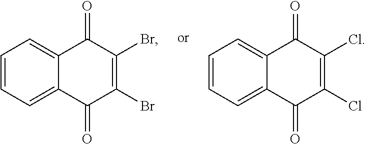 [SMILES: O=C1C(Br)=C(Br)C(=O)c2ccccc21 ; "or" ; O=C1C(Cl)=C(Cl)C(=O)c2ccccc21]

7. A methionine aminopeptidase inhibitor having the chemical structure:

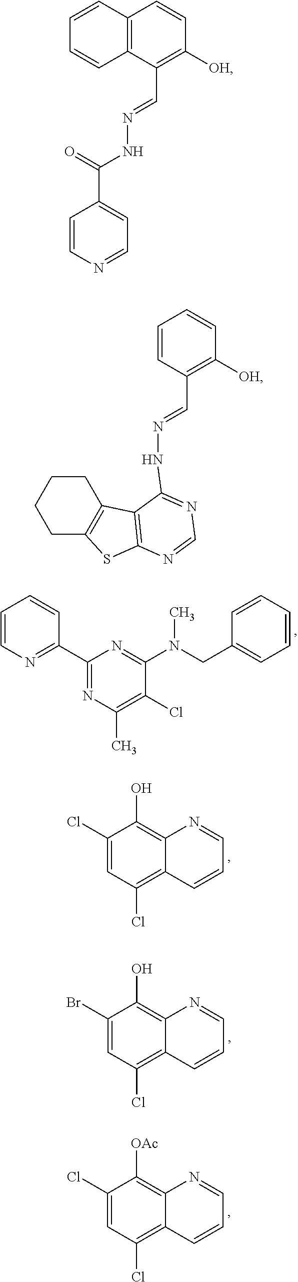

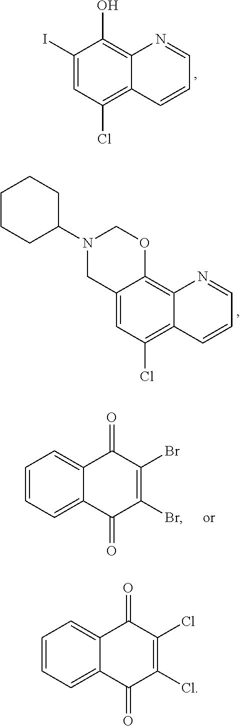

8. The methionine aminopeptidase inhibitor of claim 7, that is
5-chloro-7-iodoquinolin-8-ol;
7-bromo-5-chloroquinolin-8-ol;
5,7-dichloroquinolin-8-ol;
5,7-dichloroquinolin-8-yl acetate;
6-chloro-3-cyclohexyl-3,4-dihydro-2H-[1,3]oxazino[5,6-h]quinolin;
N-benzyl-5-chloro-N,6-dimethyl-2-(pyridin-2-yl)pyrimidin-4-amine;
N′-((2-hydroxynaphthalen-1-yl)methylene)isonicotinohydrazide;
2-{(E)-[2-(5,6,7,8-tetrahydro[1]benzothieno[2,3-d]pyrimidin-4-yl)hydrazinylidene] methyl}phenol;
2,3-dichloronaphthalene-1,4-dione; or
2,3-dibromonaphthalene-1,4-dione.


SEQUENCE LISTING (4 sequences)

SEQ ID NO: 1 - DNA, 33 bp, Artificial sequence
  LmMetAP1-Xbal sense primer to amplify LmMetAP1 gene
tctagaggatccatgccctgcgaaggctgcggc

SEQ ID NO: 2 - DNA, 41 bp, Artificial sequence
  LmMetAP1-Xbal antisense primer to amplify LmMetAP1 gene
tctagagaattctcagattttgatttcgctggggtcttcgg

SEQ ID NO: 3 - DNA, 28 bp, Artificial sequence
  forward primer to amplify N-terminal poly-His-Tag EfMAP1
gcgggatccattacattaaaatcaccac

SEQ ID NO: 4 - DNA, 26 bp, Artificial sequence
  reverse primer to amplify N-terminal poly-His-Tag EfMAP1
gcgctcgagttaataagtcaattctc